(19) United States
(12) Patent Application Publication
(10) Pub. No.: US 2018/0330801 A1
(43) Pub. Date: Nov. 15, 2018

(54) HEALTH CARE SYSTEM WITH INTERFACE FOR CHILDREN

(71) Applicant: RADICALOGIC TECHNOLOGIES INC. DBA RL SOLUTIONS, Toronto (CA)

(72) Inventors: Sanjay MALAVIYA, Mississauga (CA); Ilya MAXIMENKO, Toronto (CA); Anthony CHIARELLI, Toronto (CA); Meredith LAZOWSKI, Toronto (CA); David BACH, Toronto (CA)

(21) Appl. No.: 15/971,845

(22) Filed: May 4, 2018

Related U.S. Application Data
(60) Provisional application No. 62/504,316, filed on May 10, 2017.

(51) Int. Cl. G16H 10/20; G06F 17/24; G06F 17/27; G16H 10/60

(52) U.S. Cl. CPC G16H 10/20; G06F 17/243; G06F 3/04842; G16H 10/60; G06F 17/2705

(57) ABSTRACT

A healthcare system that includes a form engine configured to generate an interface specifically for individuals with differing cognitive abilities (e.g., children). The interface includes form fields and visual elements to receive health care and feedback data for storage in persistent data store. The visual elements can relate to requests for information and can also relate to responses to those requests. The visual elements are mapped to form fields. The healthcare system automatically populates field values corresponding to the form fields based on detected interactions with the visual elements at the interface.

### CROSS REFERENCE

This application claims all benefit, including priority to, U.S. Application No. 62/504,316, filed 10 May 2017, entitled “HEALTH CARE SYSTEM WITH INTERFACE FOR CHILDREN”, which is incorporated herein by reference.

### FIELD

The embodiments described herein relate generally to the field of healthcare systems, and more particularly, adaptive display interfaces responsive to varying levels of cognitive or linguistic ability.

### INTRODUCTION

A healthcare system can manage important health care data for different types of heath care facilities.

Healthcare environments encounter a diverse range of individuals. Individuals range across different levels of cognitive ability, physical ability, and attention spans. For example, individuals may be of different ages (toddler, teenager, young adult, adult, senior), different educational backgrounds, have difficulties with language, among others. Furthermore, linguistic and cognitive abilities may be impacted by treatments undertaken by the individuals (e.g., surgery, chemotherapy, radiotherapy, therapeutic drugs), or known characteristics of the individuals (e.g., recreational drugs, disabilities).

Healthcare practitioners and facilities solicit input from individuals pre and post procedures to obtain valuable and useful data that can be utilized to improve patient care, or to proactively identify problems that can be mitigated.

However, existing surveys for gathering input are typically not well tailored for individuals, and the rate of response is often poor, leading to sample size problems and/or data integrity issues. A further risk of poorly adapted surveys (e.g., “one size fits all”) is that individuals may simply become frustrated and provide incorrect answers in an attempt to conclude the survey quickly (e.g., picking all “c” in view of multiple choice questions).

Misleading or incorrect answers is especially damaging in view of a data-driven environment, leading to a situation where the output data from input data of questionable integrity cannot be trusted.

Manual processes are unsuitable for this role given the sheer number of surveys and forms to be customized. Further, manual customization of interfaces is untenable as the surveys often change rapidly (e.g., responsive to new legislation, operating procedures).

### SUMMARY

An improved healthcare system is described in various embodiments. The improved healthcare system is a specific improvement in the capabilities of computing devices, configured to dynamically generate and render context-specific forms based on computer-estimated cognitive, linguistic, and physical ability. An objective is to, free of human intervention, contextualize forms including interface elements and graphical user interfaces to improve response characteristics from a range of individuals.

A specific technical challenge is that healthcare systems are change-resistant as modifications to database schemas and interactions, including interfaces, require significant resources to deploy and implement. Accordingly, this is a major factor preventing the adoption of improved forms that are contextualized based on the cognitive levels of individuals, leading to reduced survey completion and/or sub-optimal results. Healthcare applications and systems are often instantiated and configured based on a static schema.

The system that is proposed is intended to provide a useful tool for patient experience officers in hospitals to collect feedback from patients and their family members and caregivers, while avoiding the need to modify how applications interact with a backend data storage.

The system allows for patients of different ages and cognitive abilities to access a common survey and respond to it using controls and interaction paradigms that are most appropriate and easy to use for them. In essence, a survey with a set of questions configured by a patient experience officer can be present and interacted with in a variety of different ways for different people. The tool controls the rendering of improved user interfaces for electronic devices. The interfaces are improved, especially for provisioning to those individuals who may have limitations from cognitive or linguistic perspectives.

An innovative data structure approach is described using a specific improvements to metabases that allows the taxonomy to be expanded where necessary to accommodate the variations in questions that can be posed based on differing cognitive levels. A flexible metaschema is utilized that is memory efficient (expanded only when necessary).

A specific data structure is described in some embodiments that provides improved flexibility relative to manual approaches where data linkages are maintained and traversed between form elements and alternates/variants thereof automatically based on a determined cognitive level of the individual. Where the survey or form is being administered on a graphical user interface in a sequential or stepwise series of interface screens, the form elements may be refactored as the questions are answered, based on a continuously determined cognitive level. “On the fly” adjustments may thus be automatically made if the initial determination was incorrect or inaccurate. The user interfaces are specifically and automatically adapted based on a common core of objects such that the survey or form may be centrally administered and modified, yet tailored when rendered for the individual users.

In some embodiments, additional pre-loading and pre-adaptation of graphical user interface elements is conducted by traversing the data structure and the linkages. In these embodiments, the adapted graphical user interface elements for downstream questions are pre-loaded into memory to improve computer performance such that the survey questions are responsively rendered without undue processing delays. This may be particularly useful where the modified graphical user interface elements may otherwise take additional processing time to prepare as compared with presentment of an un-tailored survey or form.

In accordance with one aspect, there is provided a healthcare system comprising a processor and persistent data store with instructions to configure the processor to generate an interface for children. The persistent data store stores one or more specially configured data structures which represent different permutations of forms and/or form elements, including different modalities and question mechanisms available.

The healthcare system receives computer instructions requesting the provisioning of a survey or form for one or more individuals. The healthcare system includes a cognitive level determination engine configured to estimate an cognitive level for the one or more individuals, the cognitive level determination engine configured, in some embodiments, to interface with electronic health records (EHR) databases, internal healthcare facility records, laboratory information records, among other data. The cognitive level determination engine processes the data to determine an initial cognitive level estimation, which in some embodiments, is a standardized metric or data value.

A form generation engine is configured to receive a request for generating the survey or the form, and in conjunction with the initial cognitive level estimation, dynamically generates a cognitive/linguistic level-tailored form based on the initial cognitive level estimation. In some embodiments, the form is a linked set of data objects that may be provided in the form of a data object container. In alternate embodiments, the form is a set of memory location pointers that indicate memory locations of form elements that may be invoked or called upon. A data object may include a linkage to a memory location of a current interface control/form element to be rendered, as well as a linkage to a memory location of a next interface control/form element to be rendered. In some embodiments, a pre-loading mechanism is used to dynamically prepare and pre-render a next interface control/form element so that it is more responsive to loading. This improvement is particularly useful where the form elements require additional processing time to customize or tailor in view of the estimated cognitive level metric.

Each data object may have linkages to other data objects to indicate an expected pathway or approach through specific interactive graphical user interface form elements. These linkages may also be responsively modified as the cognitive level estimation shifts during question answering, responsive to a continuously monitored cognitive level. The set of memory location pointers, in some cases, may include multiple potential next location pointers that may be selected based on tracked characteristics of the user's response (e.g., time elapsed prior to answering, verbosity of response).

A graphical user interface is rendered at a computing device. The computing device includes client computing devices, such as smart phones, tablets, personal computers, among others. The graphical user interface includes fields and visual elements to receive health care and feedback data for the persistent data store, and is configured to be controlled for rendering different visual elements and fields responsive to the estimated cognitive level.

In some embodiments, a data management component is configured to receive the health care and feedback data and store health care record entries for the health care and feedback data in the persistent data store. The data management component can use the mapping to store and update the health care record entries with the health care and feedback data.

In some embodiments, a rules engine is configured to identify a set of rules stored in the persistent data store, each rule having a trigger and an action, the trigger relating to the healthcare data in the persistent data store and the action relating to the visual elements. The form engine is configured to update the form interface to display the visual elements to receive the health care and feedback data.

In some embodiments, there is provided a computer-implemented method for dynamically generating an electronic form for receiving electronic data relating to a health care event at a user interface component, the electronic form adapted to provide a flexible meta-schema allowing a new or modified taxonomy to be adopted or accommodated without modifications to applications that process the electronic form. The method can involve: providing a healthcare event management system including a storage device and a processor, the storage device having a metabase associated therewith to maintain the meta-schema and the processor configured to provide a form engine and a system interface; establishing a communication link between the system interface and the user interface component over a network; receiving, at the system interface from the user interface component via the network, a request to generate the electronic form using the form engine; providing a dictionary of field objects in the metabase associated with the storage device of the healthcare event management system, wherein an instance of a field object is a form field, and wherein the form field is configured to receive a field value, the metabase structuring the dictionary of field objects into corresponding meta-tables and establishing meta-relations defining parent-child relationships between nodes representing the one or more field objects of the metabase, the meta-relations being maintained as corresponding foreign keys used to reference the corresponding field objects and the corresponding meta-tables, the metabase configured to have a dynamic number of columns that is dynamic according to a number of field objects in the dictionary; adding one or more additional user-defined field objects in the metabase; automatically maintaining, by the metabase, electronic representations of relationships between the field objects and the form fields, the one or more additional user-defined field objects representing the new or modified taxonomy, the automatic maintaining including: establishing, by the metabase, one or more new columns corresponding to the one or more additional user-defined field objects; and establishing new meta-relations defining the one or more additional user-defined field objects as new child nodes to corresponding parent nodes of the one or more additional user-defined field objects; generating the electronic form, using the form engine configured by the processor of the healthcare event management system, wherein the electronic form includes an ordered collection of form fields instantiated using the field objects, the ordered collection based at least on the relationships stored in the metabase including at least the meta-tables, the meta-relations, and the new meta-relations, the ordered collection of form fields being a set of visual elements; controlling the display to automatically return and display, from the system interface to the user interface component via the network, the set of visual elements, wherein each visual element corresponds to a potential field value associated with the corresponding field object used to instantiate a corresponding form field; defining, on the storage device using the metabase of the healthcare event management system; receiving, at the system interface, a selected visual element from the user interface component via the network, the selected visual element being from the set of visual elements; and controlling the electronic form using the set of visual elements based at least on the relationships stored in the metabase to adopt or accommodate the new or modified taxonomy.

Many further features and combinations thereof concerning embodiments described herein are contemplated.

The improved healthcare system of some embodiments is a special purpose machine that is configured for incorporation into a data center, interconnecting with a message bus or other communication framework to receive requests to generate surveys or forms, and to retrieve and transform data extracted from a backend data structure to modify presentment of questions, including selecting different formats for questions, different answer types, different modalities upon which to present questions, different characteristics of presentment (e.g., fonts, themes, font sizes, colors), among others.

The special purpose machine is an improved computational tool that automatically adapts form elements and presentment characteristics responsive to computer-based determinations extracted from healthcare data sets. An improved data structure backend is used in some embodiments that incorporates a specially configured metabase engine for interactions with a special purpose metabase storing meta-relations and linkages between data elements to flexibly adapt the healthcare data sets.

The system also includes components and mechanisms to create a workflow that allows a patient experience office to send out surveys to specific groups of patients/caregivers when a specific trigger event occurs, allowing them to collect information from the patient closest to the point of truth or time at which the survey response are most valuable.

Where the improved healthcare system is a special purpose machine, an example embodiment includes a specific hardware computer server appliance that is a rack-mounted device that is used as an intermediary device that intercepts survey requests, controls the customization of forms, and outputs modified forms as containers or data structure objects. The specific hardware computer server appliance has onboard memory, processors, and non-transitory computer readable media stored thereon.

In an aspect, there is provided a computer system for dynamically generating an electronic form for receiving electronic data relating to a health care event, the electronic form adapted to provide a flexible meta-schema allowing an expanded taxonomy to be adopted or accommodated without modifications to applications that process the electronic form, the system including: a user interface component on a connected device configured to receive a request to create the form; a storage device having a metabase associated therewith to maintain the meta-schema; and at least one processor configured to execute instructions to provide a form engine and a system interface.

The form engine is configured to receive a data set representative of one or more electronic health records associated with an individual, determine an estimated cognitive level of the individual by parsing the data set, and provide a healthcare event management system including a storage device and a processor, the storage device having a metabase associated therewith to maintain the meta-schema and the processor configured to provide a form engine and a system interface.

The form engine establishes a communication link between the system interface and the user interface component over a network, receives, at the system interface from the user interface component via the network, a request to generate the electronic form using the form engine, and stores, in the storage device, a dictionary of field objects in the metabase associated with the storage device of the healthcare event management system, wherein an instance of a field object is a form field, and wherein the form field is configured to receive a field value, the metabase structuring the dictionary of field objects into corresponding meta-tables and establishing meta-relations defining parent-child relationships between nodes representing the one or more field objects of the metabase, the meta-relations being maintained as corresponding foreign keys used to reference the corresponding field objects and the corresponding meta-tables, the metabase configured to have a dynamic number of columns that is dynamic according to a number of field objects in the dictionary.

The form engine, to adapt to an expanded taxonomy, is configured to add one or more additional user-defined field objects in the metabase to accommodate the expanded taxonomy capturing field object variations representative of variations of field objects that correspond to a plurality of cognitive levels and to automatically maintain, on the metabase, electronic representations of relationships between the field objects and the form fields, the one or more additional user-defined field objects representing the expanded taxonomy, the automatic maintaining including: establishing, by the metabase, one or more new columns corresponding to the one or more additional user-defined field objects; and establish new meta-relations defining the one or more additional user-defined field objects as new child nodes to corresponding parent nodes of the one or more additional user-defined field objects; generating the electronic form, using the form engine configured by the processor of the healthcare event management system, wherein the electronic form includes an ordered collection of form fields instantiated using the field objects, the ordered collection based at least on the relationships stored in the metabase including at least the meta-tables, the meta-relations, and the new meta-relations, the ordered collection of form fields being a set of visual elements.

The form engine then controls, for rendering, the electronic form using the set of visual elements based at least on the relationships stored in the metabase to adopt or accommodate the expanded taxonomy; and controls the display to automatically return and display, from the system interface to the user interface component via the network, the set of visual elements, wherein each visual element corresponds to a potential field value associated with the corresponding field object used to instantiate a corresponding form field responsive to the estimated cognitive level of the individual selected from the plurality of cognitive levels.

The form engine receives, at the system interface, a selected visual element from the user interface component via the network, the selected visual element being from the set of visual elements.

In another aspect, the expanded taxonomy includes modifications to the set of visual elements of the electronic form.

In another aspect, the modifications to the set of visual elements of the electronic form includes at least one of a displayed font size, an object visual factoring size, and a selected visual theme.

In another aspect, the selected visual theme is modified through a substitution of a cascading style sheet (CSS).

In another aspect, the data set representative of the one or more electronic health records associated with the individual is retrieved using a domain specific query language.

In another aspect, the form engine is further configured to: determine a group of target individuals to survey identified based on one or more common characteristics extracted from one or more electronic health records corresponding to each individual of the group of target individuals; for each individual of the group of target individuals, generate one or more individualized electronic form using the metabase; and control one or more user interface components, each corresponding to an individual of the group of target individuals, to render the one or more individualized electronic forms.

In another aspect, each of the new meta-relations includes a corresponding normalization factor, and the form engine is further configured to: receive one or more data sets representative of responses from the electronic form; traverse the metabase to identify one or more normalization factors associated with the set of visual elements utilized in generating the form; apply the one or more normalization factors to transform the one or more data sets into a normalized data set; and store the normalized data set in a data storage maintaining response data sets normalized to a pre-expansion taxonomy of field objects.

In another aspect, the electronic form comprises a set of sequential interface screens; and the form engine is further configured to: track one or more response characteristics of the individual as the interface renders each interface screens; responsive to the tracked one or more response characteristics, re-determine the estimated cognitive level of the individual; and responsive to a change in the estimated cognitive level of the individual, re-generate one or more remaining interface screens of the set of sequential interface screens of the electronic form.

In another aspect, the field object variations representative of variations of field objects that correspond to a plurality of cognitive levels include differing question strings appropriate for corresponding cognitive levels of the plurality of cognitive levels.

In another aspect, the field object variations representative of variations of field objects that correspond to a plurality of cognitive levels include differing visual elements appropriate for corresponding cognitive levels of the plurality of cognitive levels.

In another aspect, there is provided a computer-implemented method for dynamically generating an electronic form for receiving electronic data relating to a health care event at a user interface component, the electronic form adapted to provide a flexible meta-schema allowing an expanded taxonomy to be adopted or accommodated without modifications to applications that process the electronic form, the method comprising: receiving a data set representative of one or more electronic health records associated with an individual; determining an estimated cognitive level of the individual by parsing the data set; providing a healthcare event management system including a storage device and a processor, the storage device having a database associated therewith to maintain the meta-schema and the processor configured to provide a form engine and a system interface; establishing a communication link between the system interface and the user interface component over a network; receiving, at the system interface from the user interface component via the network, a request to generate the electronic form using the form engine; providing a dictionary of field objects in the database associated with the storage device of the healthcare event management system, wherein an instance of a field object is a form field, and wherein the form field is configured to receive a field value, the database structuring the dictionary of field objects into corresponding meta-tables and establishing meta-relations defining parent-child relationships between nodes representing the one or more field objects of the database, the meta-relations being maintained as corresponding foreign keys used to reference the corresponding field objects and the corresponding meta-tables, the database configured to have a dynamic number of columns that is dynamic according to a number of field objects in the dictionary; adding one or more additional user-defined field objects in the database to accommodate an expanded taxonomy capturing field object variations representative of variations of field objects that correspond to a plurality of cognitive levels; automatically maintaining, by the database, electronic representations of relationships between the field objects and the form fields, the one or more additional user-defined field objects representing the expanded taxonomy, the automatic maintaining including: establishing, by the database, one or more new columns corresponding to the one or more additional user-defined field objects; and establishing new meta-relations defining the one or more additional user-defined field objects as new child nodes to corresponding parent nodes of the one or more additional user-defined field objects; generating the electronic form, using the form engine configured by the processor of the healthcare event management system, wherein the electronic form includes an ordered collection of form fields instantiated using the field objects, the ordered collection based at least on the relationships stored in the database including at least the meta-tables, the meta-relations, and the new meta-relations, the ordered collection of form fields being a set of visual elements; controlling the display to automatically return and display, from the system interface to the user interface component via the network, the set of visual elements, wherein each visual element corresponds to a potential field value associated with the corresponding field object used to instantiate a corresponding form field responsive to the estimated cognitive level of the individual selected from the plurality of cognitive levels; controlling the electronic form using the set of visual elements based at least on the relationships stored in the database to adopt or accommodate the expanded taxonomy; and receiving, at the system interface, a selected visual element from the user interface component via the network, the selected visual element being from the set of visual elements.

Corresponding methods, processes, devices, machines, tools, and computer readable media are contemplated.

Corresponding computer readable media store machine interpretable instructions, which when executed, cause a processor to perform steps of any combination of the computer implemented methods described above. Computer readable media include solid state media, hard disk storage media, random access memories, read-only memories, among others. Persistent, non-transitory media are contemplated.

### DESCRIPTION OF THE FIGURES

Embodiments will now be described, by way of example only, with reference to the attached figures, wherein in the figures:

FIG. 1A is a block schematic of an example healthcare system according to some embodiments.

FIG. 1B is illustrative of an example special purpose machine of some embodiments.

FIG. 1C is an example diagram of an example computing device.

FIG. 2A is a block schematic of an example healthcare system according to some embodiments.

FIG. 2B is a block schematic of an approach for cognitive level determination according to some embodiments.

FIG. 3 is an example screenshot of a form interface with different visual elements.

FIG. 4 is an example list of factors or considerations for children.

FIG. 5 is example form interfaces according to some embodiments.

FIG. 6 is an example list of factors or considerations for patient experience surveys.

FIG. 7 is an example form interface with the messaging feature according to some embodiments.

FIG. 8 is an example form interface with a feedback feature according to some embodiments.

FIG. 9 is an example form interface with confirmation according to some embodiments.

FIG. 10 is an example form interface with a patient report according to some embodiments.

FIG. 11 is an example form interface with a login feature according to some embodiments.

FIG. 12 is an example form interface with a feedback feature according to some embodiments.

FIG. 13 is an example form interface with confirmation and a feedback feature according to some embodiments.

FIG. 14 is an example form interface with confirmation according to some embodiments.

FIG. 15 is an example form interface with patient activity according to some embodiments.

FIG. 16 is an example form interface for a family patient experience according to some embodiments.

FIG. 17 is an example form interface with a feedback feature according to some embodiments.

FIG. 18 is an example list of factors for people with low cognitive and age level. People with a low cognitive and age level might not be able to read or write, may need help from parents or other adults to communicate, and may have low motor skills. These factors can be used to generate a dynamic form interface suitable for a person with low cognitive and age level.

FIG. 19 is an example of a patient interacting with form interface. A young patient is using the form interface to provide feedback on a physician, the food, the bed and other features using visual elements. The patient can also submit audio feedback.

FIG. 20 is an example form interface with a feedback feature according to some embodiments. The form interface includes a visual element for a thumbs up or like and a visual element for a thumbs down or just like. The form interface includes a visual element representing a physician. The form interface can be used to provide feedback relating to the physician using the visual elements.

FIG. 21 is an example form interface with a feedback feature according to some embodiments. In this example, a patient interacts with the form interface to drag the visual element representing the physician to the visual element for like to provide feedback that the patient likes the physician. The collected feedback can be stored automatically to populate field values associated with feedback for the physician.

FIG. 22 is an example form interface with a feedback feature according to some embodiments. In this example, the form interface includes visual elements representing things that the patient liked about the health care facility experience. This can be a summary screen based on the collected feedback.

FIG. 23 is an example list of factors for people with medium cognitive and age level. People with medium cognitive and age level can be encouraged by rewards and recognition, can struggle with breaking down broad questions, and can enjoy expressing personality. These factors can be used to generate a dynamic form interface suitable for a person with medium cognitive and age level.

FIG. 24 is an example form interface with an avatar creation feature according to some embodiments. The form interface can include visual elements representing an avatar along with features for the avatar such as face, hair, nose, mouth, eyes, and years. This enables a patient to generate a virtual representation that they are comfortable or associate with.

FIG. 25 is an example form interface according to some embodiments. In this example, the form interface provides a game that can facilitate the collection of relevant data.

FIG. 26 is an example form interface according to some embodiments. In this example, the form interface provides a game to collect souvenirs.

FIG. 27 is an example form interface with a challenge feature according to some embodiments. In this example, the form interface provides a challenge to engage the user.

FIG. 28 is an example list of factors for people with high cognitive and age level. People with high cognitive and age level can develop conversation skills, can be receptive to social participation, and may overly game of find experiences juvenile.

FIG. 29 is an example form interface with a greeting feature according to some embodiments. In this example, the form interface includes a messaging and greeting feature. The greeting feature may include a question. The message feature includes the ability to drag different visual elements to represent healthcare data and responses.

FIG. 30 is an example form interface with a feedback feature according to some embodiments. In this example, the form interface includes a messaging and feedback feature. The message can include a question. The message feature includes the ability to drag different visual elements to represent feedback data.

FIG. 31 is an example form interface of the messaging feature according to some embodiments. In this example, the form interface includes a messaging feature that enables a healthcare provider to give tips to improve the health care experience. This may increase the amount of positive feedback received. The tips can be automatically generated based on collected feedback. For example, collected feedback may indicate the person does not like the food and the tip can suggest an item to try on the menu.

FIG. 32 is an example form interface with a messaging feature according some embodiments. In this example, the form interface includes a comment feature and a report feature to summarize the collected feedback.

FIG. 33 is an example form interface according to some embodiments. In this example, the form interface provides options to a patient including playing a game to collect data regarding the healthcare experience.

FIG. 34 is an example form interface according to some embodiments. In this example, the form interface includes a patient hub to find further information.

FIG. 35 is an example form interface for an administrator form interface, according to some embodiments. In this example, the form interface allows for the generation of a question generally directed to the quality of food.

FIG. 36 is an example form interface for an administrator form interface, according to some embodiments. In this example, the form interface allows for the generation of variations of a question related to food for a kids audience.

FIG. 37 is an example form interface for an administrator form interface, according to some embodiments. In this example, the form interface allows for the generation of variations of a question related to food for a teenagers audience.

FIG. 38 is an example administrator interface for tracking distribution of a survey, according to some embodiments.

FIGS. 39A and 39B are data model diagrams illustrating the improved backend table relationships that support the improved metabase of some embodiments.

### DETAILED DESCRIPTION

In some embodiments, the features utilize a specially configured metabase backend for improved computational processing efficiency. Applicant hereby incorporates by reference U.S. application Ser. No. 13/070,754, filed 24 Mar. 2011, entitled “SYSTEMS AND METHODS FOR CREATING A FORM FOR RECEIVING DATA RELATING TO A HEALTH CARE INCIDENT”.

Healthcare systems are difficult and cumbersome to manage in view of strict regulatory oversight and privacy requirements. Modifications to healthcare devices and software ecosystems are difficult to adopt as they require extensive testing and change management mechanisms to handle transitions. Accordingly, it is beneficial to design flexible and adaptive computational approaches to avoid unnecessary transitions. In some cases, improved data structures and mechanisms are utilized to facilitate dynamic approaches that allow for modified taxonomies to be adopted in relation to form fields, while maintaining what appears to be an unchanged schema to the applications and devices that interact with a database backend.

The embodiments described herein are directed specifically to computer implemented methods, devices, apparatuses, and computer readable media for adaptive display interfaces responsive to varying levels of cognitive or linguistic ability. An improved computing data architecture and backend is described in various embodiments.

In particular, an improved approach is described herein that enables flexibility in schema to handle an expanded taxonomy that is utilized to support the automatic tailoring of forms, form controls, visual elements, and interface elements such that as forms are generated for individuals to fill in (e.g., surveys), the improved data architecture and backend is able to adapt the forms, form controls, visual elements, and interface elements to match an estimated cognitive level (or linguistic level) of the individual.

This is useful, especially where individuals are children, aged, have disabilities, or have linguistic challenges. Survey responses are particularly difficult to obtain, and there are serious shortcomings with a traditional “one-size fits all” approach with conventional static surveys. Manually tailoring surveys and form information is an impractical approach that is extremely time consuming and fraught with errors. Given that significant validation processes are required to re-tool existing applications, where there are any interface changes required in how an application interacts a backend data storage, the approach is unlikely to be adopted.

A form is provided to an individual in a healthcare setting to solicit feedback in relation to some aspect to their care, treatment, or stay (“event”) at the hospital. An event could be targeted for various aspects, including, for example, patient satisfaction, patient experience, patient outcomes, patient reported outcomes, patient reported experience measures. Events may also trigger forms/surveys to be provided, for example, if an adverse event occurs (e.g., adverse drug reaction from wrong medication, a person fell). Events may be triggered even if the individual is not directly impacted, for example, family members of a person who fell and was injured. The surveys and forms can be utilized to track to see whether issues were rectified, and there may be multiple surveys and/or forms that are generated in response to an event trigger. Surveys and forms can be presented to various individuals, for example, patients, practitioners, visitors, etc.

Information from the surveys/forms is captured in a response table (e.g., JSON). The response data structure can be broadcast, for example, along a message bus to be made available to other applications within the healthcare facility. There may be actions that are triggered based on the response data structure (e.g., formal complaint). The response data structure can be processed in the aggregate to generate reports and other types of graphics or outputs.

Expanding a taxonomy of field objects, as described above, is a difficult computational task that could, absent a specially configured metabase or database structure backend acting as an intermediary, require significant changes to existing applications, devices, and software.

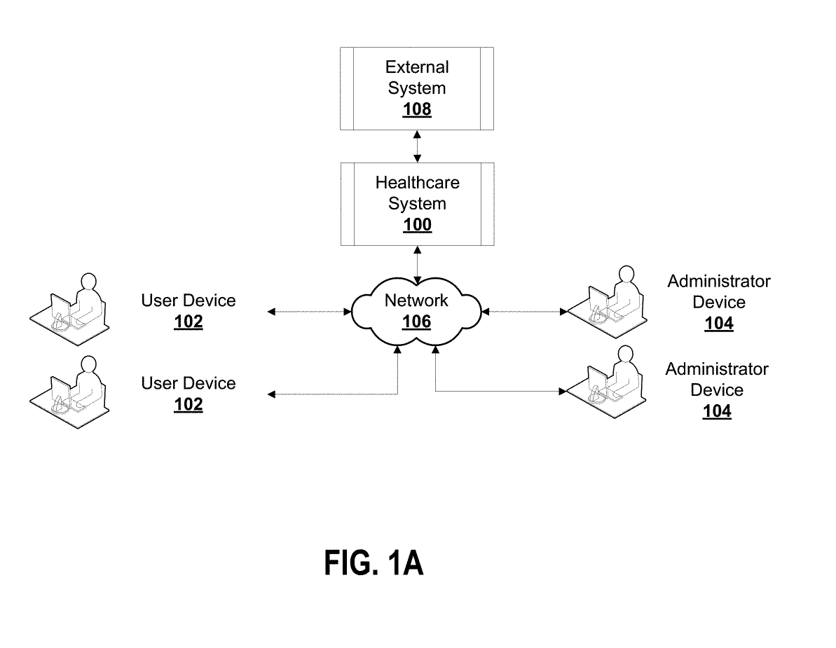

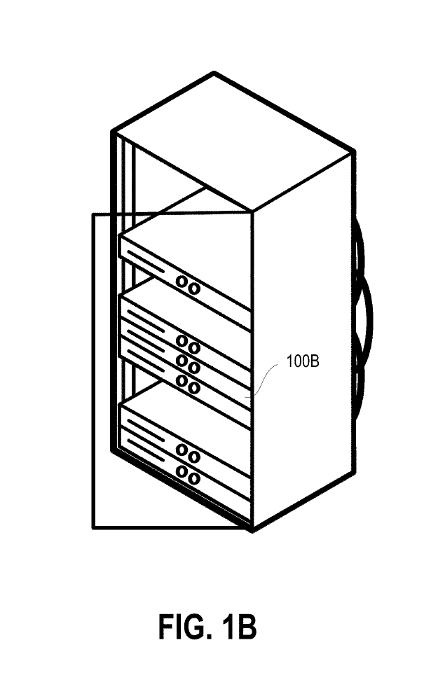

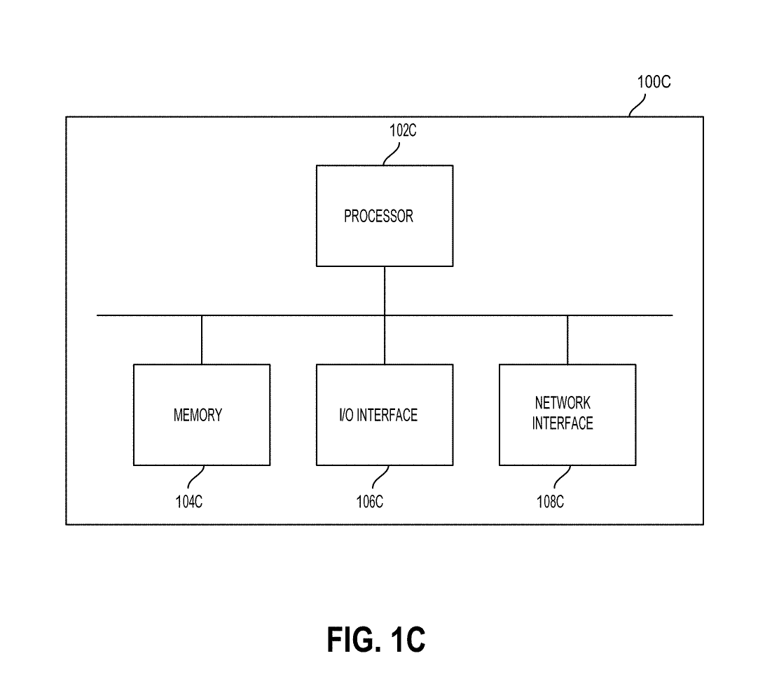

FIG. 1 is a schematic of an example healthcare system 100 according to some embodiments. The healthcare system 100 generates and controls an interface on user device 102 to display a form of form fields that include visual elements. The form of visual elements provides an alternative way to control the display and collection of healthcare data. The form of visual elements may be suitable for different individuals (e.g., children) as they may have varied cognitive skills. The form of visual elements provides an alternative way to display form fields and collect field values that represent healthcare data.

Healthcare system 100 is a specific improvement in the capabilities of computing devices, configured to dynamically generate and render context-specific forms based on computer-estimated cognitive, linguistic, and physical ability. An objective is to, free of human intervention, contextualize forms including interface elements and graphical user interfaces to improve response characteristics from a range of individuals, while avoiding the need for modifications of underlying applications and their interaction with the schema of an underlying backend database or metabase.

The healthcare system 100 connects to user device 102 and administrator device 104 in various ways, including directly coupled and indirectly coupled via the network 106. Network 106 (or multiple networks) is capable of carrying data and can involve wired connections, wireless connections, or a combination thereof. Network may involve different network communication technologies, standards and protocols. The healthcare system 100 connects to external system 108 to exchange data and commands, for example.

The healthcare system 100 includes at least one processor, memory, at least one I/O interface, and at least one network interface. The processor may be, for example, a microprocessor or microcontroller, a digital signal processing (DSP) processor, an integrated circuit, a field programmable gate array (FPGA), a reconfigurable processor, or any combination thereof. Memory may include computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM).

Each I/O interface enables a computing device to interconnect with one or more input devices, such as a keyboard, mouse, camera, touch screen and a microphone, or with one or more output devices such as a display screen and a speaker. Each network interface enables healthcare system 100 to communicate with other components, to exchange data with other components, to access and connect to network resources, to serve applications, and perform other computing applications by connecting to a network (or multiple networks) capable of carrying data. The healthcare system 100 is operable to register and authenticate user device 102 and administrator device 104 (using a login, unique identifier, and password for example.

A data management component 200 is configured to receive the health care and feedback data and store health care record entries for the health care and feedback data in the persistent data store. The data management component 200 can use the mapping to store and update the health care record entries with the health care and feedback data.

A rules engine is configured to identify a set of rules stored in the persistent data store, each rule having a trigger and an action, the trigger relating to the healthcare data in the persistent data store and the action relating to the visual elements. The form engine is configured to update the form interface to display the visual elements to receive the health care and feedback data.

An improved data structure backend is used in some embodiments that incorporates a specially configured metabase engine for interactions with a special purpose metabase storing meta-relations and linkages between data elements to flexibly adapt the healthcare data sets.

Where the improved healthcare system is a special purpose machine 100B, an example embodiment includes a specific hardware computer server appliance that is a rack-mounted device that is used as an intermediary device that intercepts survey requests, controls the customization of forms, and outputs modified forms as containers or data structure objects. The specific hardware computer server appliance has onboard memory, processors, and non-transitory computer readable media stored thereon.

The system 100 also includes components and mechanisms to create a workflow that allows a patient experience office to send out surveys to specific groups of patients/caregivers when a specific trigger event occurs, allowing them to collect information from the patient closest to the point of truth or time at which the survey response are most valuable. The surveys can be distributed by an alert engine.

FIG. 1B is illustrative of an example special purpose machine of some embodiments. The improved healthcare system 100B is a special purpose machine that is configured for incorporation into a data center, interconnecting with a message bus or other communication framework to receive requests to generate surveys or forms, and to retrieve and transform data extracted from a backend data structure to modify presentment of questions, including selecting different formats for questions, different answer types, different modalities upon which to present questions, different characteristics of presentment (e.g., fonts, themes, font sizes, colors), among others. The special purpose machine 100B is an improved computational tool that automatically adapts form elements and presentment characteristics responsive to computer-based determinations extracted from healthcare data sets.

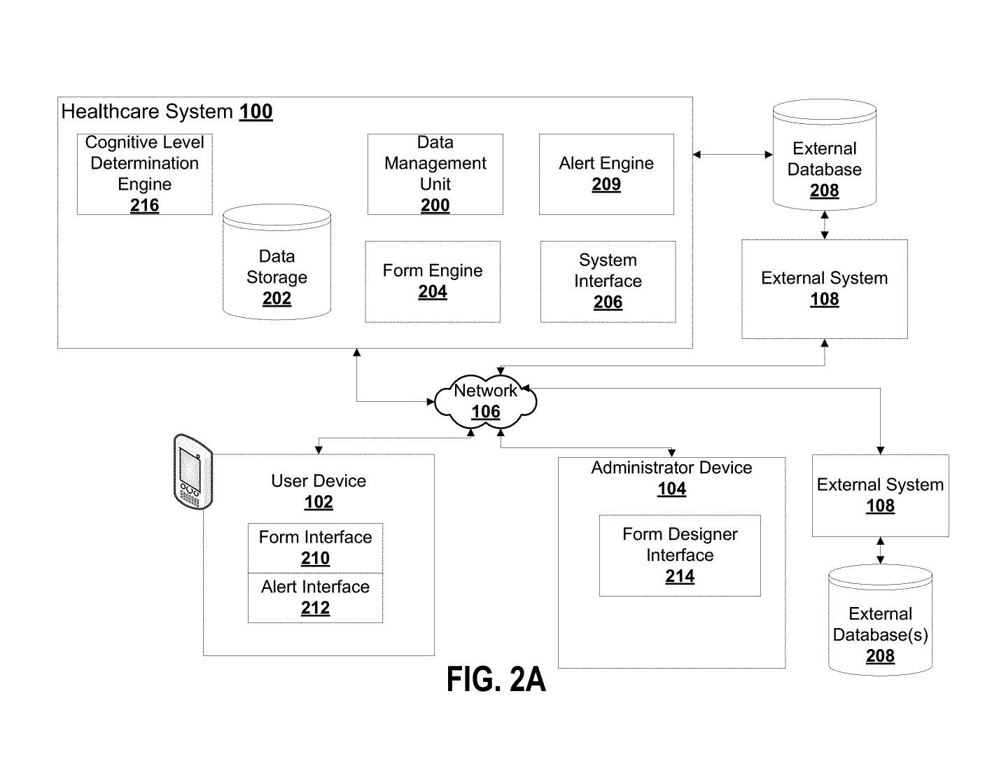

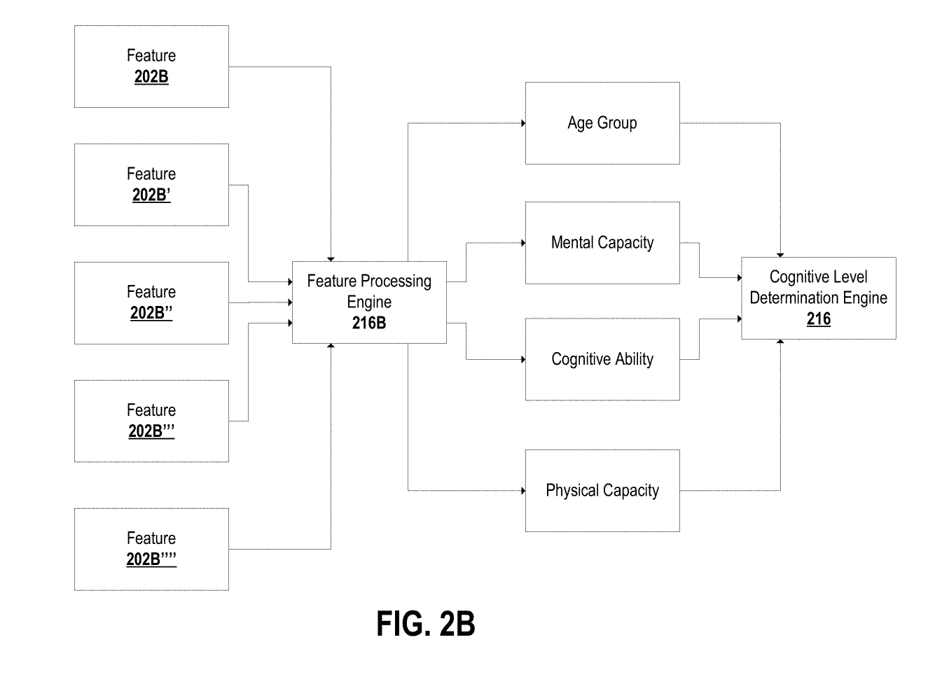

FIG. 1C is an example diagram of an example computing device 100C. Example computing device 100C can be utilized to implement various embodiments of the system 100, including those components and modules shown in FIG. 2. Computing device 100C includes processor 102C, which operates in conjunction with memory 104C, an input/output interface 106C, and a network interface 108C.

FIG. 2A is a schematic of an example healthcare system 100 according to some embodiments. The healthcare system 100 is configured for dynamically generating an electronic form for receiving electronic data relating to a health care event, the electronic form adapted to provide a flexible meta-schema allowing an expanded taxonomy to be adopted or accommodated without modifications to applications that process the electronic form, the system including: a user interface component on a connected device configured to receive a request to create the form; a storage device having a metabase associated therewith to maintain the meta-schema; and at least one processor configured to execute instructions to provide a form engine and a system interface.

Healthcare system 100 includes data storage, data management unit, form engine, alert engine, and system interface 206. The user device 102 includes a form interface 210 and alert interface 212. The administrative device 104 includes a form designer interface 214.

The form engine 204 is configured to receive a data set representative of one or more electronic health records associated with an individual, determine an estimated cognitive level of the individual by parsing the data set through cognitive level determination engine 216. The data set representative of the one or more electronic health records associated with the individual can, in some embodiments, be retrieved using a domain specific query language.

The form engine 204 establishes a communication link between the system interface 206 and the user interface component over a network 106 and receives, at the system interface 206 from the administrator interface component on administrator device 104 via the network 106, a request to generate the electronic form using the form engine.

When administrator device 104 utilizes form designer interface 214 to design a form having variations adapted for individuals of varying cognitive levels, the form engine 204 is configured to store and maintain, in the data storage 202, a dictionary of field objects in the metabase associated with the storage device of the healthcare event management system.

An instance of a field object is a form field, and the form field is configured to receive a field value, the metabase structuring the dictionary of field objects into corresponding meta-tables.

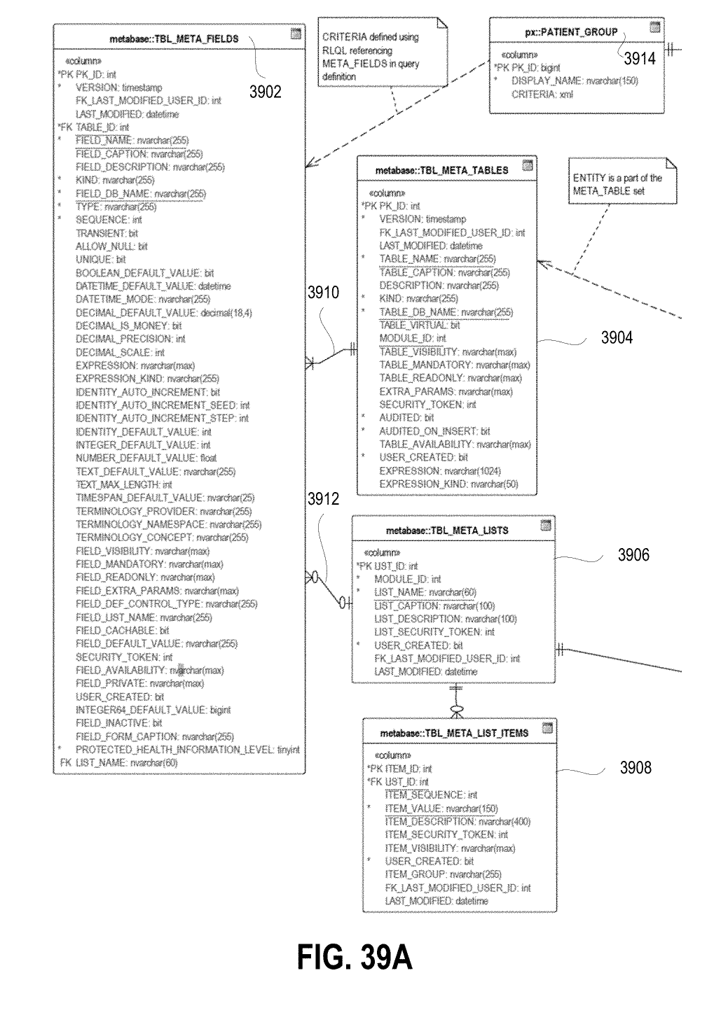

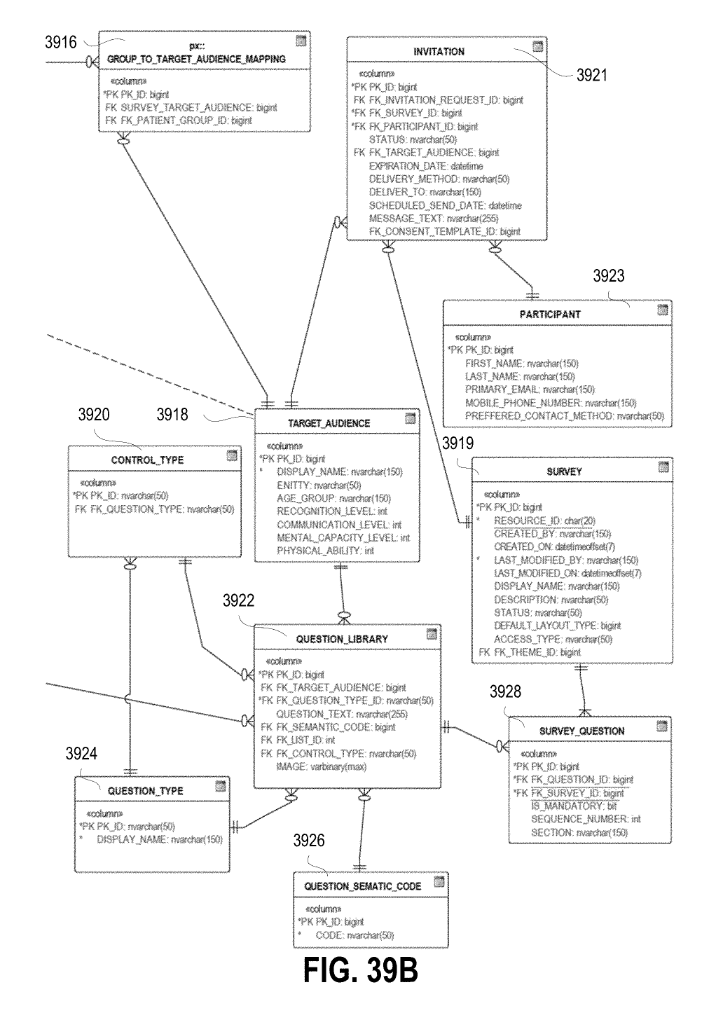

Meta-relations are established defining parent-child relationships between nodes representing the one or more field objects of the metabase. The meta-relations, in some embodiments, are maintained as corresponding foreign keys used to reference the corresponding field objects and the corresponding meta-tables, the metabase configured to have a dynamic number of columns that is dynamic according to a number of field objects in the dictionary. An example mapping of meta-relations and meta-tables is shown at FIGS. 39A and 39B.

The use of meta-relations defined accordingly enables the metabase to more flexibly expand to accommodate the expanded taxonomy that results due to the variations accommodating form element differences in cognitive levels. A benefit is that applications can maintain interoperability with a data backend without explicit configuration to adapt to the modified taxonomy (that is expanded to accommodate question variations).

The form engine 204, to adapt to the expanded taxonomy, is configured to add one or more additional user-defined field objects in the metabase to accommodate the expanded taxonomy capturing field object variations representative of variations of field objects that correspond to a plurality of cognitive levels and to automatically maintain, on the metabase, electronic representations of relationships between the field objects and the form fields, the one or more additional user-defined field objects representing the expanded taxonomy, the automatic maintaining including: establishing, by the metabase, one or more new columns corresponding to the one or more additional user-defined field objects; and establish new meta-relations defining the one or more additional user-defined field objects as new child nodes to corresponding parent nodes of the one or more additional user-defined field objects; generating the electronic form, using the form engine configured by the processor of the healthcare event management system, wherein the electronic form includes an ordered collection of form fields instantiated using the field objects, the ordered collection based at least on the relationships stored in the metabase including at least the meta-tables, the meta-relations, and the new meta-relations, the ordered collection of form fields being a set of visual elements.

The form engine 204 then controls, for rendering, the electronic form using the set of visual elements based at least on the relationships stored in the metabase to adopt or accommodate the expanded taxonomy; and controls the display to automatically return and display, from the system interface to the user interface component via the network, the set of visual elements, wherein each visual element corresponds to a potential field value associated with the corresponding field object used to instantiate a corresponding form field responsive to the estimated cognitive level of the individual selected from the plurality of cognitive levels.

The form engine 204 receives, at the system interface 206, a selected visual element from the user interface component via the network, the selected visual element being from the set of visual elements. The expanded taxonomy includes modifications to the set of visual elements of the electronic form, including, for example, a displayed font size, an object visual factoring size, and a selected visual theme.

Refactoring of visual elements is possible, for example, to show objects in different selected visual themes modified, for example, through a substitution of a cascading style sheet (CSS). Other variations include simpler questions being rendered, simpler response interactive elements (e.g., radio buttons instead of free text), among others. Variations may also be made in relation to color, font size, font selection, positioning on a screen, transparency effects, among others. For example, for children, there may be a child-friendly theme that is presented, along with questions with simple language that are used to convey a same or similar meaning.

Referring to FIG. 2B, the form engine 204 is further configured to determine a group of target individuals to survey identified based on one or more common characteristics extracted from one or more electronic health records corresponding to each individual of the group of target individuals. Health information may be extracted through features 202B-202B″″, which are feature sets derived from raw electronic health record data. A feature processing engine 216B processes these to extract information such as age group, mental capacity, cognitive ability, and physical ability. A score may be assigned for each of these, and an aggregate score may be generated that is ultimately processed by the cognitive level determination engine 216 to generate the estimated cognitive level.

For each individual of the group of target individuals, the form engine 204 is configured to generate one or more individualized electronic form using the metabase for distribution through alert engine 209, for example. When the forms are rendered, form engine 204 controls the rendering of the one or more user interface components on user device 102, each corresponding to an individual of the group of target individuals, to render the one or more individualized electronic forms.

Each of the new meta-relations may include a corresponding normalization factor. The normalization factor is used to ensure that received data is readily comparable. For example, if every form is individually tailored, potentially differently based on cognitive level, it may be useful to ensure that the received data can be normalized to map to a baseline data set (e.g., the parent survey with no variations). The form engine 204 may receive one or more data sets representative of responses from the electronic form and traverse the metabase or database of data storage 202 to identify one or more normalization factors associated with the set of visual elements utilized in generating the form (e.g., by inspecting the parent-child linkages).

The form engine 202 can then apply the one or more normalization factors to transform the one or more data sets into a normalized data set and store the normalized data set in a data storage 202 or a data warehouse that maintains response data sets normalized to a pre-expansion taxonomy of field objects. The data storage 202 or a data warehouse can then be used to generate reports or other views for display, for example, on administrator device 104.

The electronic form of some embodiments includes a set of sequential interface screens; and the form engine is further configured to: track one or more response characteristics of the individual as the interface renders each interface screens; responsive to the tracked one or more response characteristics, re-determine the estimated cognitive level of the individual; and responsive to a change in the estimated cognitive level of the individual, and re-generate one or more remaining interface screens of the set of sequential interface screens of the electronic form.

An improved metabase maintained on data storage 202, in particular, is well suited to this task as metabase technology described in various embodiments herein is designed with an expandable and modifiable taxonomy encapsulated in a dynamic set of columns that track field objects through a set of parent-child relationships, meta-relation linkages.

Meta-tables and meta-relations can be utilized to track and store the expanded taxonomy, and may be called upon to render different interface elements based on the determination of the estimated cognitive level. The meta-relations and meta-tables can be traversed in accordance with the determination of the estimated cognitive level. Appropriate visual elements may thus be rendered by the application, without any need to modify the application to accept the updated taxonomy.

The backend database or metabase stored and maintained on data storage 202 is adapted to store a specific survey comprising sets of visual interface elements (graphical elements, including placement/positioning of same on a screen, thematic controls), question types, response options (e.g., radio buttons, pick lists, free text inputs). Variations of survey elements are included in the backend database or metabase.

Variations are stored in accordance with an innovative approach to configuring the backend database or metabase such that stored linkages within the backend database or metabase are utilized to render the adaptation of the form/survey elements to be computationally transparent to the existing applications. In other words, the existing applications operate a same way but the backend database or metabase is configured to selectively apply differing visual sets of elements estimated to be appropriate for the cognitive level of the individual.

A specific data structure is described in some embodiments that provides improved flexibility relative to manual approaches where data linkages are maintained and traversed between form elements and alternates/variants thereof automatically based on a determined cognitive level of the individual.

Data storage 202 stores healthcare data, rules, visual elements, form objects, form fields, field values and other data. Data management unit 200 processes rules to evaluate triggers and execute actions. The actions can relate to retrieving or generating visual elements for form interface 210. Data storage 202 may store other information, such as electronic health record data (EHR data), laboratory data, procedure data, among others. The data sets may be stored, for example, in the form of HL7 or other standards data, and may include admission discharge transfer (ADT) data, patient identities, visit information, among others.

The form engine 204 controls the administration of the form on a user device 102, which includes (or has residing upon) form interface 210 and alert interface 212. Form engine 204, in some embodiments, renders a survey or administers the survey on a graphical user interface in a sequential or stepwise series of interface screens.

These sequential or stepwise series of interface screens may include a subset of form elements that are displayed and responses are tracked as answers are received. Where there is a sequential or stepwise series of interface screens, there is an opportunity for the system 100 of some embodiments to modify an estimated cognitive level. For example, if health care records indicate that an individual had recently received drowsy inducing medication and is answering a survey, the initial estimated cognitive level may be low.

However, during the course of responding, the form engine 204 may track the individual's response characteristics (e.g., using sensors located on user device 102), which indicate that the individual is accurately answering questions in a very fast (e.g., less than 1 second per screen) and responsive manner (e.g., fulsome full text answers being provided). The initial estimated cognitive level may thus be too low and may be re-adjusted upwards such that the form survey cognitive level is dynamically adjusted for rendering visual elements and controls on subsequent screens (e.g., the questions become more fulsome, and more complex variations are presented instead, appropriate to the newly estimated cognitive level).

Similarly, the reverse is possible, where the system 100 tracks difficulties in answering (e.g., individual is skipping questions, answering “c” to all of the multiple choice answers, individual is taking a long time per question), and may, responsive to these tracked characteristics, reduce the estimated cognitive level leading to downstream shifts in form generation for subsequent form screens. Re-factoring form screens is thus possible, and desirable, especially where records are unreliable, or over/under estimate cognitive function (e.g., may be the individual metabolizes a drug faster than others).

Data storage 202 stores a more generalized survey/set of form questions and controls, which have expanded variations thereof that are utilized to vary the survey or set of form questions for different cognitive levels. The user interfaces are specifically and automatically adapted based on the common core of objects such that the survey or form may be centrally administered and modified, yet tailored when rendered for the individual users.

Form engine 204 is configured for pre-loading and pre-adaptation of graphical user interface elements is conducted by traversing the data structure and the linkages.

In these embodiments, the adapted graphical user interface elements for downstream questions are pre-loaded into memory on user device 102 to improve computer performance such that the survey questions are responsively rendered without undue processing delays. This may be particularly useful where the modified graphical user interface elements may otherwise take additional processing time to prepare as compared with presentment of an un-tailored survey or form. The metabase and meta-relations are invoked in graphical user interface elements based on the estimated cognitive level such that appropriate visual elements will be rendered.

The healthcare system 100 receives computer instructions requesting the provisioning of a survey or form for one or more individuals from an administrator device 104. The administrator device 104 may, for example, been utilized by an administrator to prepare a survey or form having various variations based on cognitive level, using form designer interface 214.

The survey or form elements are provided to data storage 202, which may include a specifically configured metabase or database that adapts the variations and stores them in the form of child-parent relationships. In some embodiments, the relationships and linkages between a parent question/visual elements and its variations may further include header information indicative of a normalization metric that is utilized later to transform received responses normalized to match responses directed to the parent question/visual elements. In these embodiments, the transformed received responses can then be stored as data sets alongside received responses where individuals were only asked the parent questions and compared accordingly without further data processing.

The healthcare system 100 includes a cognitive level determination engine 216 configured to estimate an cognitive level for the one or more individuals, the cognitive level determination engine configured, in some embodiments, to interface with electronic health records (EHR) databases, internal healthcare facility records, laboratory information records, among other data (e.g., stored on external systems 108 and/or external databases 208 and processed using data management unit 200). In alternate embodiments, the cognitive level is set by administrator device 104 and associated with each individual. In alternate embodiments, cognitive level determination engine 216 configured to estimate an cognitive level by way of an individual's age.

The cognitive level determination engine 216 processes the data to determine an initial cognitive level estimation, which in some embodiments, is a standardized metric or data value. In an embodiment, the cognitive level is determined primarily by age, and then adjusted based on secondary factors, such as the presence of drugs or procedures known to affect cognitive ability, time of day, among others.

The form generation engine 204 is configured to receive a request for generating the survey or the form, and in conjunction with the initial cognitive level estimation, dynamically generates a cognitive/linguistic level-tailored form based on the initial cognitive level estimation.

In some embodiments, the form is a linked set of data objects that may be provided in the form of a data object container. In alternate embodiments, the form is a set of memory location pointers that indicate memory locations of form elements that may be invoked or called upon. A data object may include a linkage to a memory location of a current interface control/form element to be rendered, as well as a linkage to a memory location of a next interface control/form element to be rendered. Each data object may have linkages to other data objects to indicate an expected pathway or approach through specific interactive graphical user interface form elements. These linkages may also be responsively modified as the cognitive level estimation shifts during question answering, responsive to a continuously monitored cognitive level. The set of memory location pointers, in some cases, may include multiple potential next location pointers that may be selected based on tracked characteristics of the user's response (e.g., time elapsed prior to answering, verbosity of response).

A graphical user interface is rendered at a computing device such as user device 102 by way of form interface 210. The computing device includes client computing devices, such as smart phones, tablets, personal computers, among others.

The graphical user interface includes fields and visual elements to receive health care and feedback data for the persistent data store, and is configured to be controlled for rendering different visual elements and fields responsive to the estimated cognitive level. The form engine 204 interacts with a form interface 210 to render forms having form fields and corresponding form field values to receive and transmit healthcare data. The form fields include visual elements to receive field values representing healthcare and feedback data. The form engine 204 generates a mapping between the form fields and visual elements. When a visual element is used to receive field values the form engine 204 and the data management unit 200 can use the mapping to link the field values to the form fields in data storage 202. The form interface 210 interacts with system interface 206 to create or update healthcare data in database 202 based on data received using the visual elements. The form engine 204 interacts with a form designer interface 216 to create, customize and configure forms, form fields, and visual elements rendered by the form interface 210.

Healthcare system 100 and form engine 204 may integrate features of the healthcare workflow system described in U.S. Provisional Patent Application No. 62/353,707 filed Jun. 23, 2017, this contents of which is hereby incorporated by reference.

The form engine 204 that interacts with a client-side component (e.g. form interface 210) to render forms (with form fields for receiving form values) on user device 102. The form may operate in submission mode (the creation of new files) and management modes (the modification of files to add additional form fields and field values, adding follow-up actions to be taken on a file, for example). As an illustrative example, the form engine 204 may display data from a patient file stored in database 202 on user device 102 as a set of form fields represented as visual elements (e.g. as part of form interface 210). User device 102 can submit commands to modify data in the patient file through form fields and visual elements. When rendering a form, the form engine 204 may intercept and execute form rules that are configured by administrator device 104 through a form designer interface 214.

The form rules can map or link visual elements to form fields. In some embodiments, form rules provide instructions to make sections and fields of a form visible or hidden. Form rules may be configured using form designer 214 and stored in database 202 for access by form engine 204.

In some embodiments, the form engine 204 interacts with form engine application programming interfaces (APIs) that provide definitions, constraints and rules, visual elements and the like. The form engine 204 APIs interpret, parse and send defined form rules to form engine 204 and user device 102 to render forms with visual elements corresponding to form fields. The form interface 210 that is controlled by the form engine 204 may be designed by administrator device 104 through the form designer interface 214.

The form designer interface 214 is used to design forms and visual elements and configure form rules including customized or pre-defined logical expressions to configure the display, visibility, editability (e.g. write privileges) and other features of form components. As an illustrative example, a form rule may toggle the visibility of a form field or tab in a form. In this example, administrator device 104 may configure form rules through the form designer interface 214 that user device 102 is part of a group to permit writing to a patient file using form fields. As another example, administrator device 104 may configure form rules through the form designer interface 214 that user device 102 does not have write privileges to modify a particular form field corresponding to data entry in patient files.

As a result, the form engine 204, through its form engine APIs may process form rules that have been created by the administrator device 104 and hide this field from the certain user device 102 who do not have sufficient privileges to modify the data contained in the field. In some embodiments, the form engine 204 may synchronously process and execute form rules specific to a user device 102. Form rules may monitor interaction in real time by the user device 102 with a particular form. Further, a form rule can transmit data to an auxiliary API to execute predefined programmatic instructions.

The form engine 204 is configured to identify a set of rules stored in the persistent data store, each rule having a trigger and an action, the trigger relating to the healthcare data in the persistent data store and the action relating to the visual elements. The form engine 204 is configured to update the form interface to display the visual elements to receive the health care and feedback data.

The form engine 204 detects events based on received or updated healthcare data from the form interface 210. The events may also relate to interactions with the visual elements of the form interface to 10. The form engine 204 stores event entries for the detected events to an events queue stored in data storage 202. Each rule can have a trigger and an action relating to the healthcare data in the data storage 202 or an event entry, for example.

Healthcare system 100 can have an alert engine 208 to transmit alert notifications to an alert interface 212 on user device 102 based on rules and events. The alerts can also include visual elements as an alternative mechanism to provide notification and alerts as part of an alert interface to 12. Accordingly the alert engine 208 can use rules to link alerts to one or more visual elements to be displayed as part of the alert interface 212.

In some embodiments, healthcare system 100 is configured for dynamically generating a form interface 210 for receiving electronic data relating to a health care event at a user device 102. The electronic form interface 210 can implement a flexible meta-schema allowing a new or modified taxonomy to be adopted or accommodated without modifications to applications that process the electronic form interface 210. The data management unit 200 and the storage device 202 can implement a metabase associated therewith to maintain the meta-schema. Further details on the meta-schema and the metabase are provided in U.S. application Ser. No. 14/295,637 and U.S. Pat. No. 8,781,852, the contents of which are hereby incorporated by reference.

A form engine 204, system interface 206 and a form interface 210 establish a communication link between the system 100 and the user device 102 over a network.

The form engine 204 receives from the user device 102 via the network 106, a request to generate the electronic form interface 210 using the form engine 204. The storage device 202 can provide a dictionary of field objects in the metabase. An instance of a field object is a form field, and the form field is configured to receive a field value. The form field can be represented in form interface to 10 as a visual element to receive a corresponding field value. The metabase structuring the dictionary of field objects into corresponding meta-tables and establishing meta-relations defining parent-child relationships between nodes representing the one or more field objects of the metabase. The meta-relations can be maintained as corresponding foreign keys used to reference the corresponding field objects and the corresponding meta-tables. The metabase is configured to have a dynamic number of columns that is dynamic according to a number of field objects in the dictionary.

The healthcare system 100 can add one or more additional user-defined field objects in the metabase. The data storage 202 automatically maintains, by the metabase, electronic representations of relationships between the field objects and the form fields, the visual elements, the one or more additional user-defined field objects representing the new or modified taxonomy. The metabase can establish one or more new columns corresponding to the one or more additional user-defined field objects, and can establish new meta-relations defining the one or more additional user-defined field objects as new child nodes to corresponding parent nodes of the one or more additional user-defined field objects.

The form engine 204 can generate the form interface 210 to include an ordered collection of form fields instantiated using the field objects, the ordered collection based at least on the relationships stored in the metabase including at least the meta-tables, the meta-relations, and the new meta-relations, the ordered collection of form fields being a set of visual elements. The form engine 204 can control a display of the user device 102 to automatically return and display, from the system interface 206 to the form interface 210 via the network, the set of visual element. Each visual element can correspond to a potential field value associated with the corresponding field object used to instantiate a corresponding form field. The data storage 202 can define using the metabase a selected visual element from the form interface to 10 via the network, the selected visual element being from the set of visual elements. The form engine 204 can control the form interface to 10 using the set of visual elements based at least on the relationships stored in the metabase to adopt or accommodate the new or modified taxonomy.

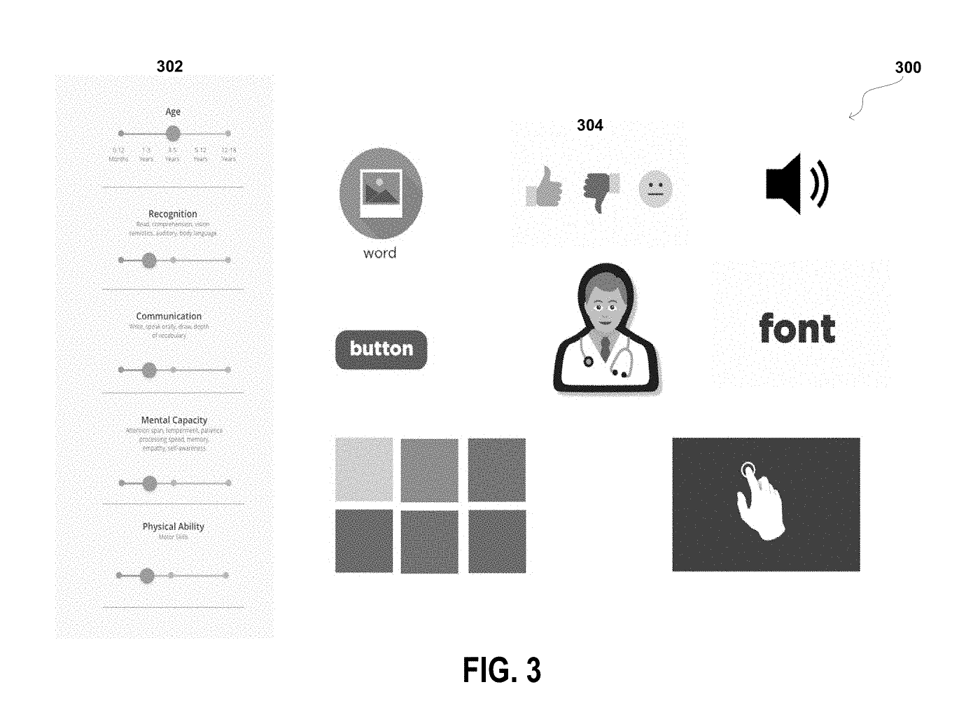

FIG. 3 is an example form interface 300 with different visual elements 302, 304. A first set of visual elements 30 representing factors for a patient including: age; recognition an comprehension of words, imagery, symbols, audio and body language; communication ability to writing, speaking, drawing and so on; mental capacity such as attention span, memory, temperament, empathy, self-awareness, and so on; and physical ability such as motor skills. The form interface 300 generates and selects visual elements 302, 304 in a dynamic way based on these factors to provide a dynamic interactive experience tailored to a patient with a particular set of factors. A second set of visual elements 304 include buttons, indicia, images, feedback, emoticons, and so on. The visual elements provide kid friendly feedback mechanisms. A one-size-fits-all approach does not work for all interfaces as different cognitive factors and age levels need to be considered. The visual elements 302, 304 of form interface 300 can help visualize information for different age and cognitive levels including low, medium, and high. The example form interface 300 can be for a low cognitive level. There can be use of more imagery through visual elements 302, 304 as opposed to words that cannot be read or understood. The form interface 300 can trigger audio to help communicate with children that cannot read. The visual elements 302, 304 can be in a primary colour palette for example. The visual elements 302 and 304 provide a simple and universal imagery to avoid distraction and confusion. The form interface 300 is triggered using simple gesture interactions such as tax or dregs. Big buttons can be used with large click areas to facilitate interactivity.

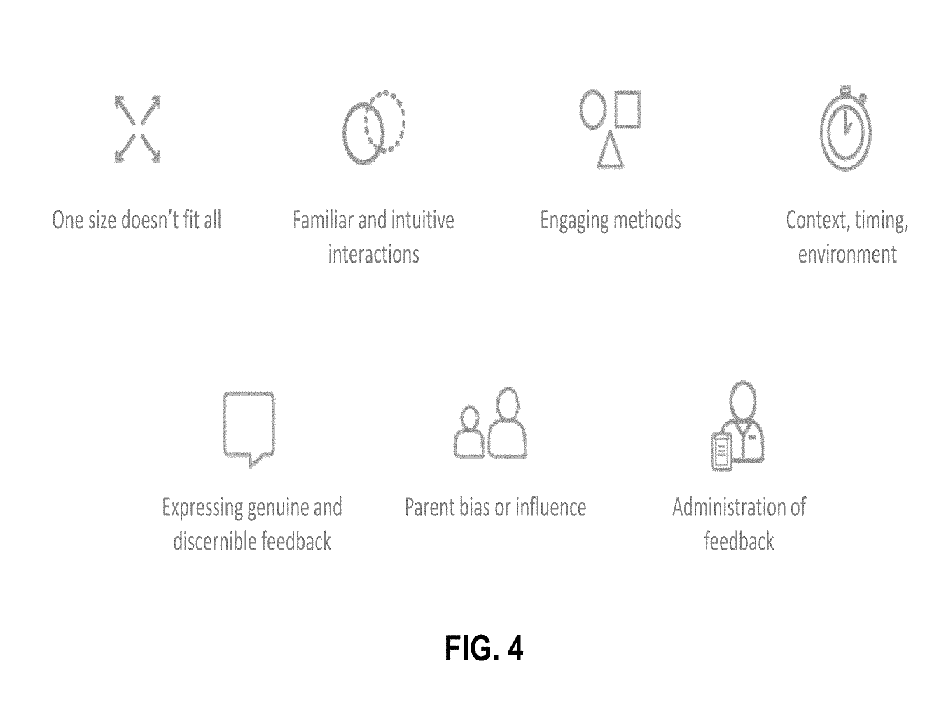

FIG. 4 is an example list of factors or considerations for children. There is not a one size fits all solution as children range from a toddler with different factors and needs to a teenager with specific factors and needs including different cognitive levels periods. Healthcare system 100 uses methods that are familiar and intuitive for children to facilitate interaction with the form interface. Healthcare system 100 uses methods that are engaging such as games. Contacts, timing and environment influence the feedback experience. For example if feedback is only requested at the end of a visit it may be difficult to recall specific memories. The visual elements can be used to help kids express genuine and discernible feedback. If the experience is too game like then this may lead to expressing imaginative feedback and not true feedback, for example. The visual elements can solicit feedback directly from kids without the influence of parents which may remove bias. Healthcare system 100 improves the experience for the person administering the feedback by making it easier for children to provide an express feedback.

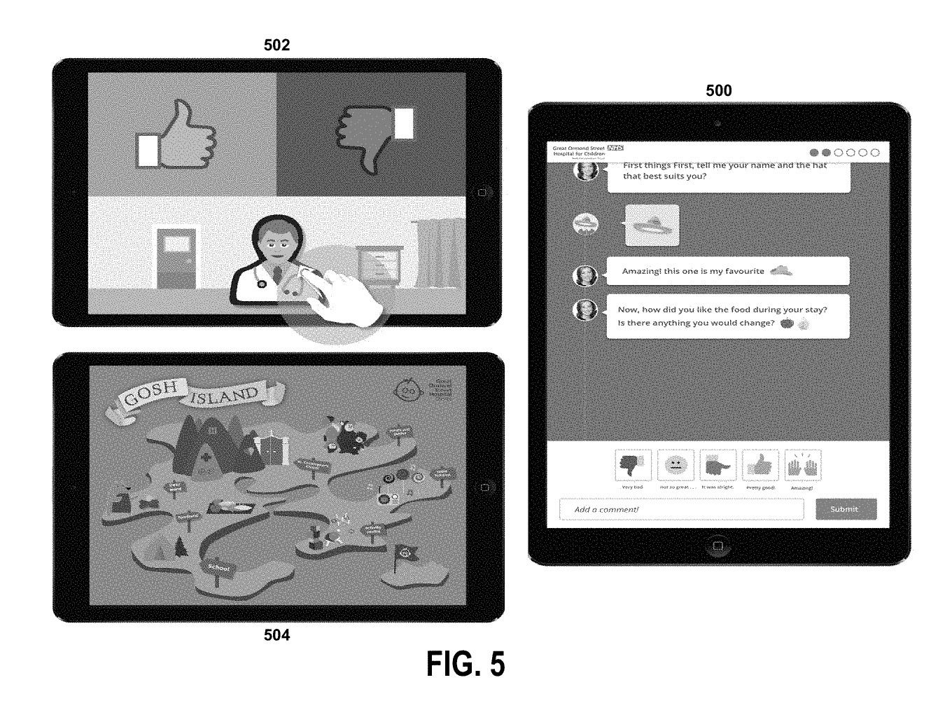

FIG. 5 is example form interfaces 500, 502, 504 according to some embodiments. A form interface 500 can provide a messaging and feedback feature using visual elements. A form interface 502 can provide a feedback feature using visual elements. A form interface 504 can provide a game feature.

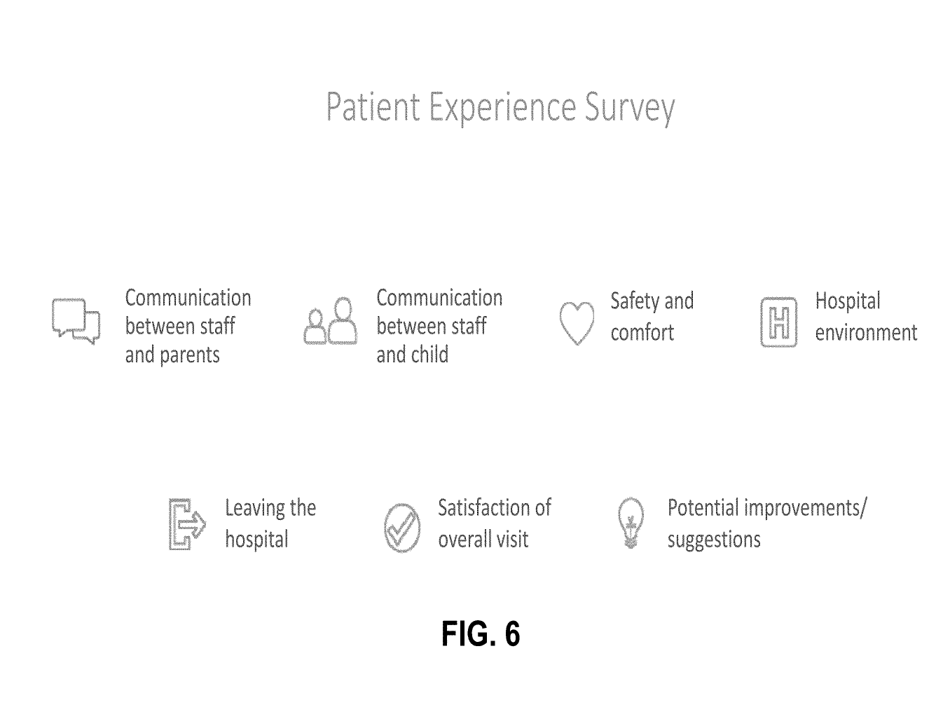

FIG. 6 is an example list of factors or considerations for patient experience surveys. Healthcare system 100 can improve the patient experience and an understanding of the experience through feedback. Healthcare system 100 can provide the opportunity for children to say what they liked or disliked about their experience at a healthcare facility. Healthcare system 100 can provide children and young people a voice to provide feedback on their experience. This might provide empowerment. Healthcare system 100 can provide an improved patient experience survey to facilitate communication between staff and parents in communication between staff and children. For example, the feedback can indicate if staff were informative and explain why certain treatment was needed and the patient was being listened to. The feedback can indicate the safety and comfort of the patient and the availability of staff. The feedback can indicate the hospital environment including cleanliness, hygiene compliance, noise levels, food quality, and entertainment and activities. The feedback can relate to hospital dispatch including being informed about medicine and who to contact for further questions. The feedback can indicate the satisfaction of the overall visit and if they would recommend the healthcare facility to family and friends. The feedback can provide potential improvements and suggestions.

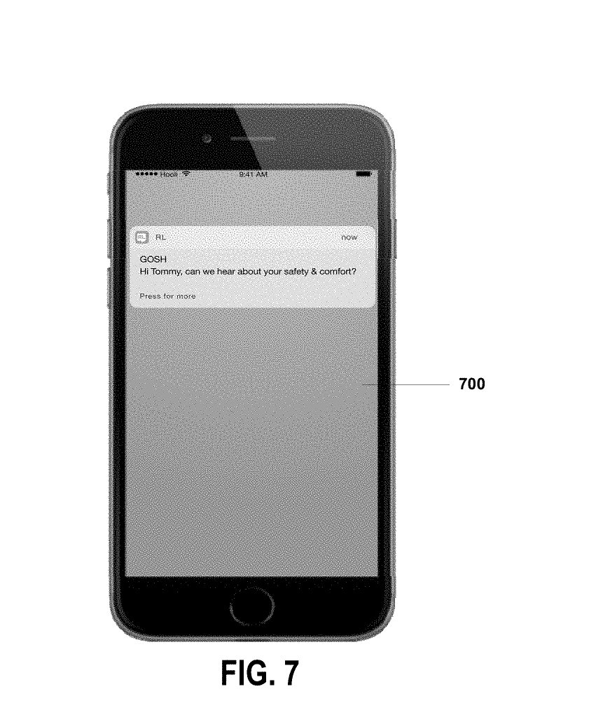

FIG. 7 is an example form interface 700 with the messaging feature according to some embodiments. The form interface 700 can be displayed on user device 102 as hospital initiated feedback. The messaging feature can include our request for feedback about safety and comfort for example. The user device 102 can be a smart phone and the messaging feature can include a request to participate in a survey to help the patient experience team understand how the patient is feeling about his safety and comfort at the healthcare facility.

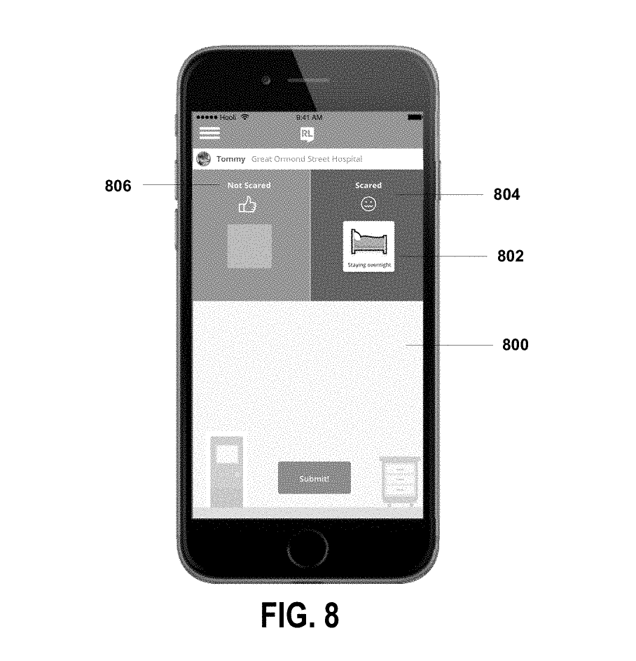

FIG. 8 is an example form interface 800 with a feedback feature according to some embodiments. The form interface 800 provides a feedback survey to help the patient participate and a sorting activity relating to aspects of a healthcare facility. The survey can relate to fears relating to the healthcare facility. For example the patient can be requested to sort hospital items according to what they are scared of and not scared of. For example the patient can drag a picture of a needle into the scared section 804 because they are scared of injections. As another example of patient can drag a picture of a bed 802 to the scared section 804 to indicate they are scared of staying overnight. The pictures or images are example visual representations. There may also be a not scared section 806.

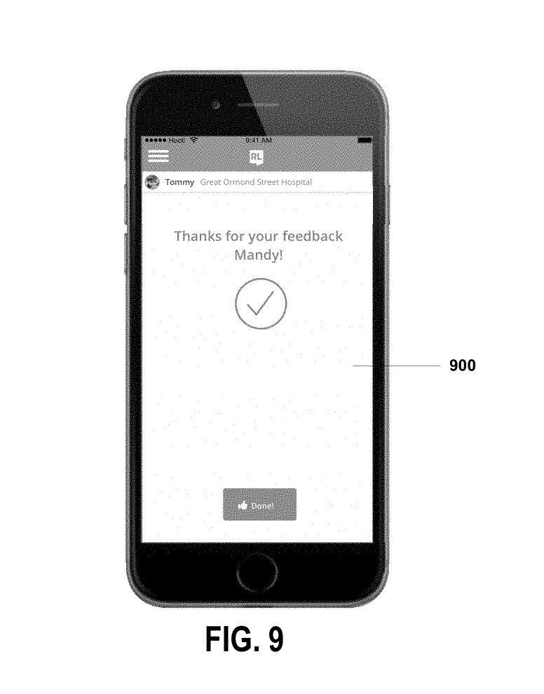

FIG. 9 is an example form interface 900 with confirmation according to some embodiments. The form interface 900 can provide questions for parents. The parent can be asked some supporting questions relating to the feedback received from the patient for example.

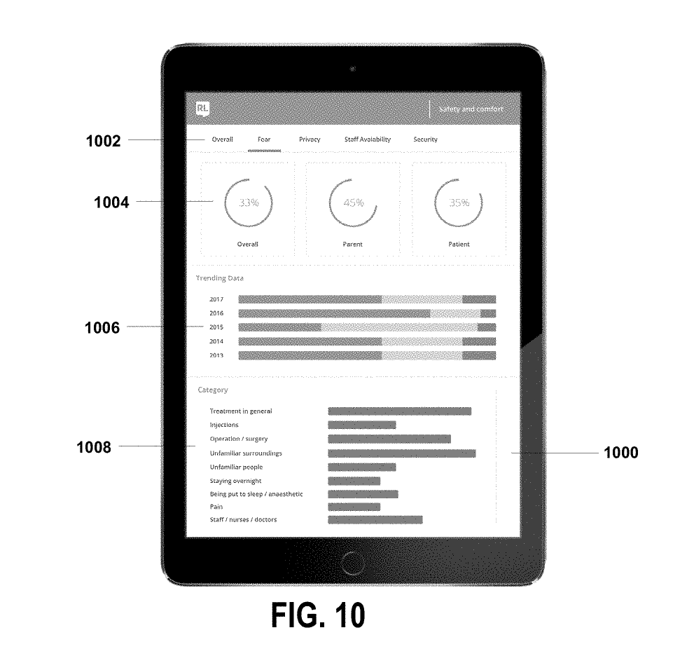

FIG. 10 is an example form interface 1000 with a patient report according to some embodiments. The form interface 1000 can provide an administrative dashboard to generate patient reports to help the patient experience team understand feedback relating to the healthcare facility. The form interface 1000 can provide different pages 1002 including an overall report, a fear report, a privacy report, a staff availability report, and a security report. The form interface 1000 can provide a visual representation of an overall feedback score a parent feedback score and the patient feedback score. The form interface 1000 can include one or more charts 1006 two indicate trending data analytics. The form interface 1000 can include a category chart 1008 for different aspects of feedback.

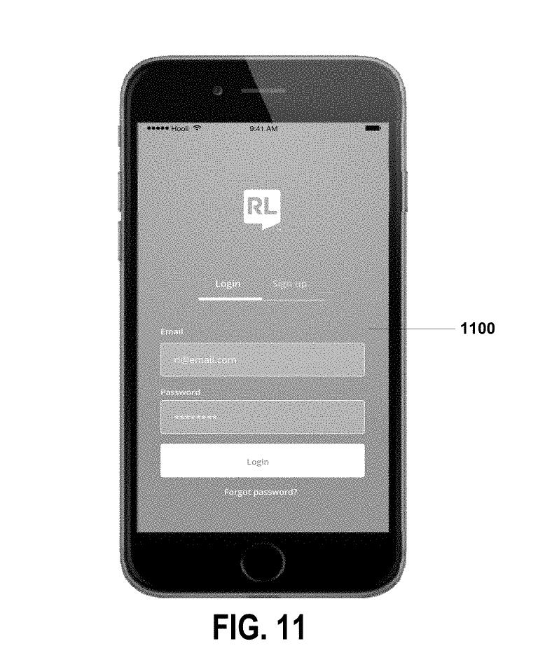

FIG. 11 is an example form interface 1100 with a login feature according to some embodiments. The form interface 1100 is an example of family initiated feedback. Sometimes families want to provide feedback on an ongoing basis rather than waiting for staff to provide a survey.

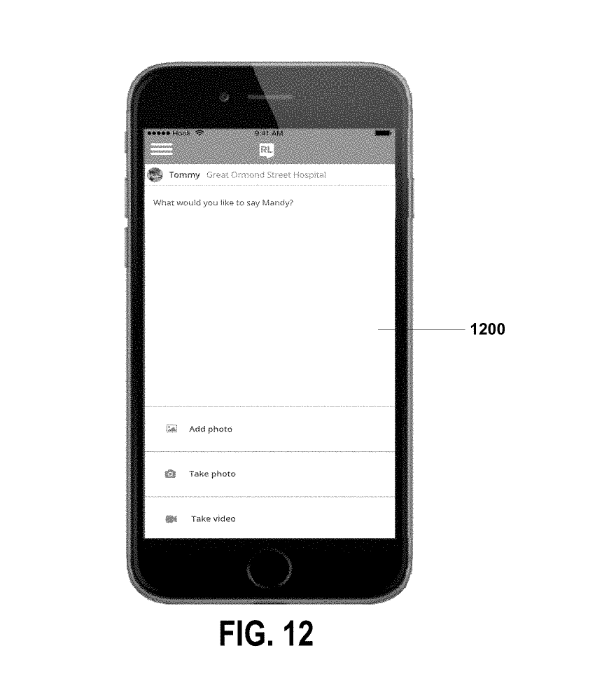

FIG. 12 is an example form interface 1200 with a feedback feature according to some embodiments. The form interface 1200 shows login screen to select the patient (a child) and indicate a virtual check in at a healthcare facility. There can be a list of feedback categories were each feedback category selectable. In this example the patient can select the safety and comfort back category from the list of feedback categories. The form interface 1200 provides options for how to provide feedback including taking a photo, taking a video, or providing written feedback.

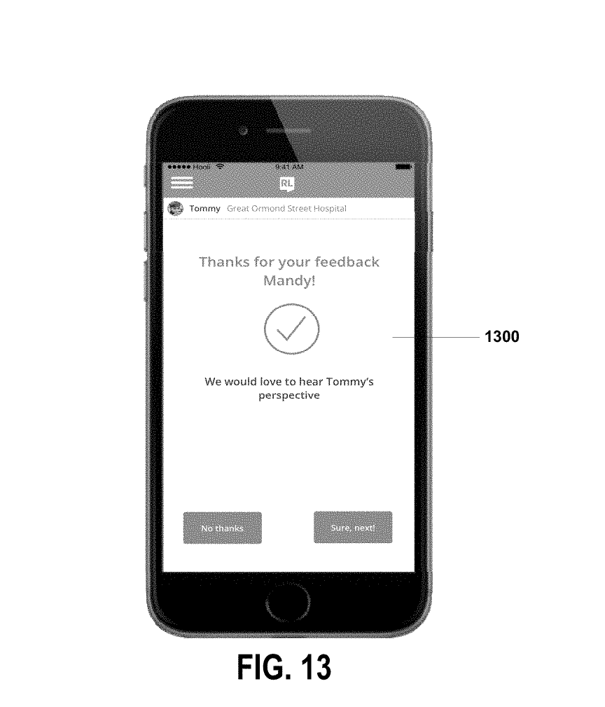

FIG. 13 is an example form interface 1300 with confirmation and a feedback feature according to some embodiments. The form interface 1300 provides confirmation that an adult has provided feedback. The form interface 1300 provides a prompt to receive feedback from the child who is the patient in this example.

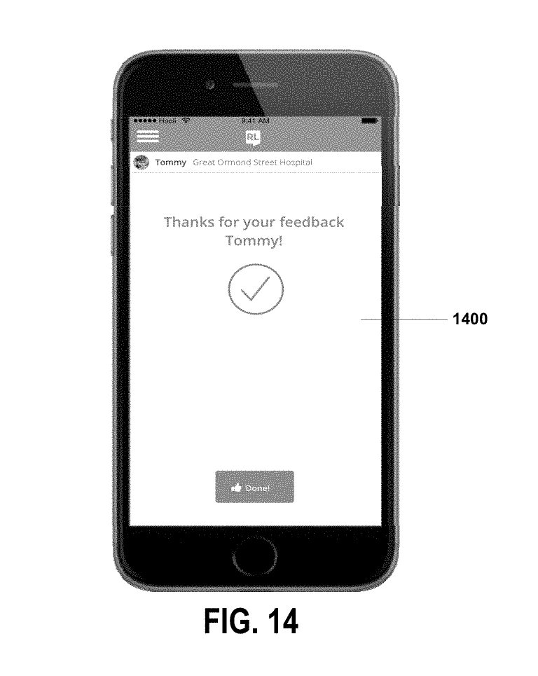

FIG. 14 is an example form interface 1400 with confirmation according to some embodiments. The form interface 1400 provides confirmation that the child patient has provided feedback. For example the child can use a form interface 1400 to create a picture about his experience or add music to illustrate his experience.

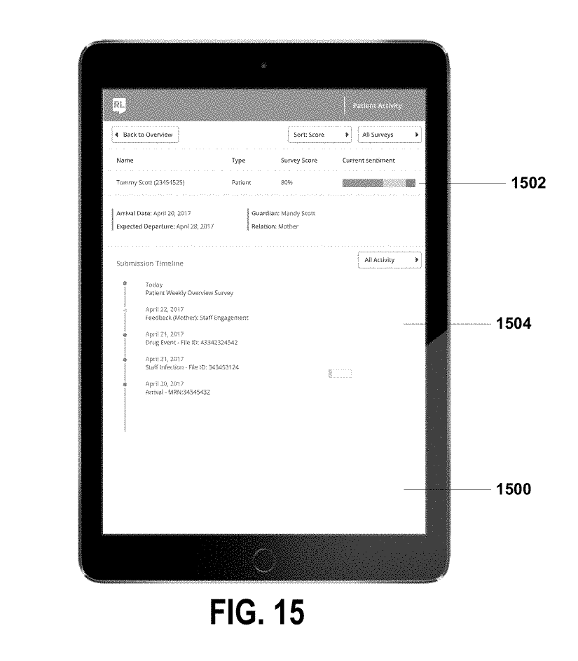

FIG. 15 is an example form interface 1500 with patient activity according to some embodiments. The form interface 1500 includes an administrative patient feedback summary view. From the administrative view, the form interface 1500 provides a patient feedback summary for a patient experience team to view. The form interface 1500 includes a current sentiment score 1502. The form interface 1500 includes submission timeline 1504 to display a timeline of all submitted feedback whether it is hospital initiated or family initiated feedback. The form interface 1500 outlines the story of the patient experience with the healthcare facility including positive and negative feedback.

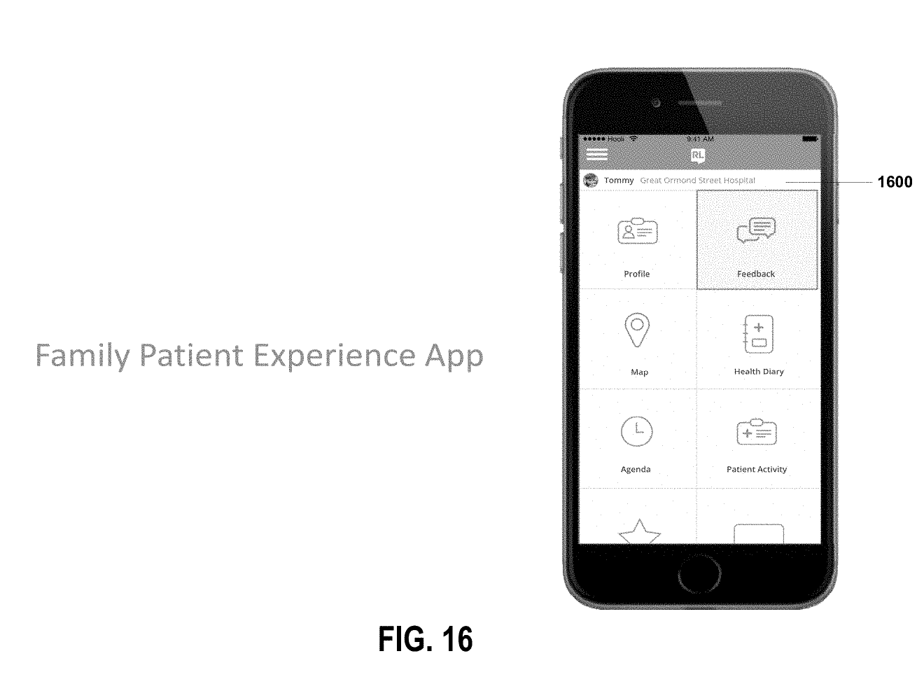

FIG. 16 is an example form interface 1600 for a family patient experience according to some embodiments. The form interface 1600 can be to implement a family patient experience at. The form interface 1600 can include different component such as a profile feature, feedback feature, Matt feature, health diary feature, agenda feature and a patient activity feature, for example. Accordingly, form interface 1600 can include multiple features in addition to feedback features. Each feature can be linked to a set of visual elements to collect relevant data and automatically map the collected data to form fields and field values. The features themselves can be example visual elements. The agenda can provide a dynamic and real-time agenda for procedures or medications.

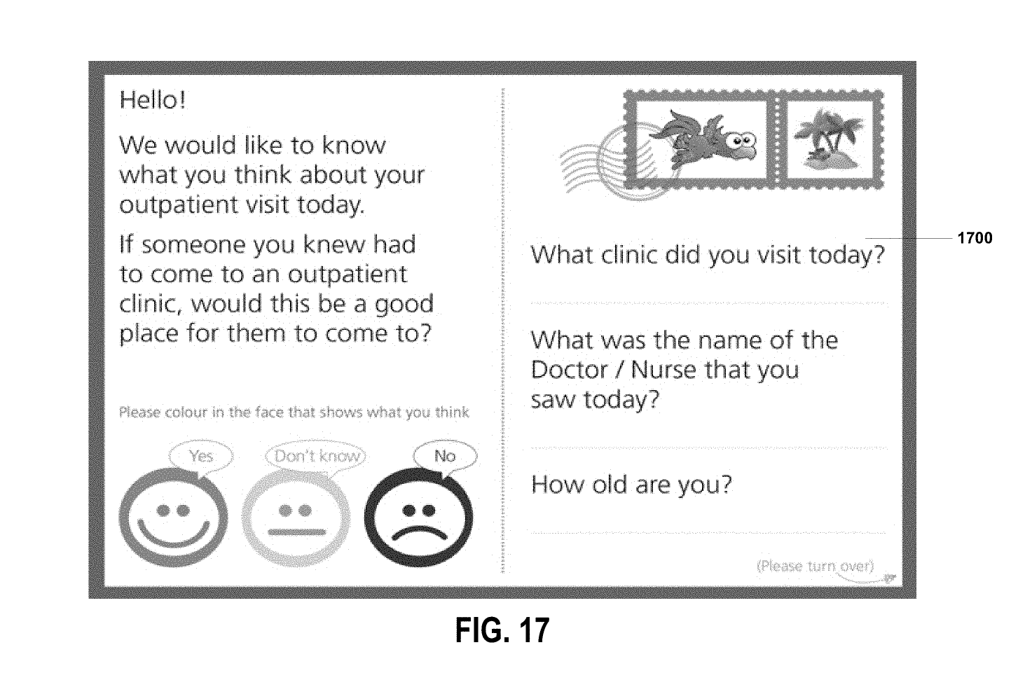

FIG. 17 is an example form interface 1700 with a feedback feature according to some embodiments. The form interface 1700 includes different visual elements to receive feedback from the patient along with the simple questionnaire.

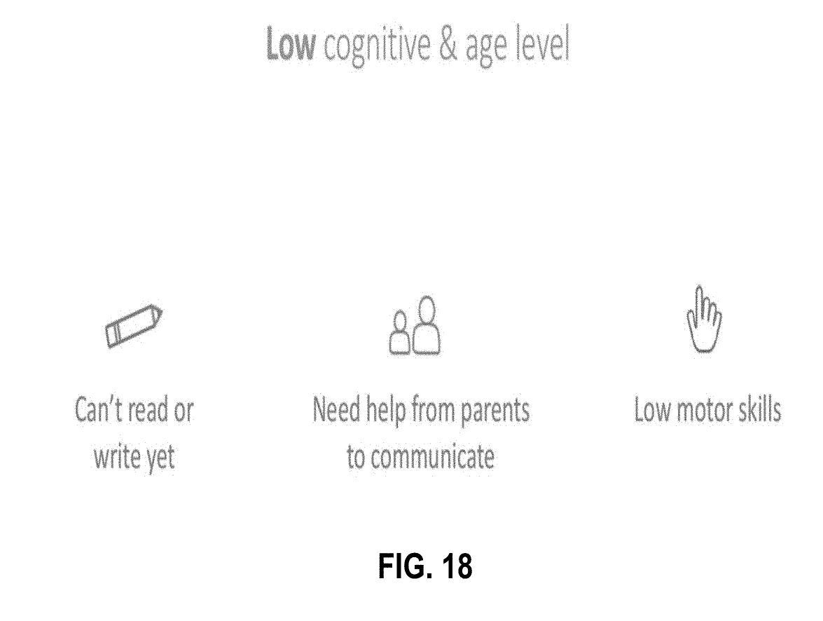

FIG. 18 is an example list of factors for people with low cognitive and age level. People with a low cognitive and age level might not be able to read or write, may need help from parents or other adults to communicate, and may have low motor skills. These factors can be used to generate a dynamic form interface suitable for a person with low cognitive and age level.

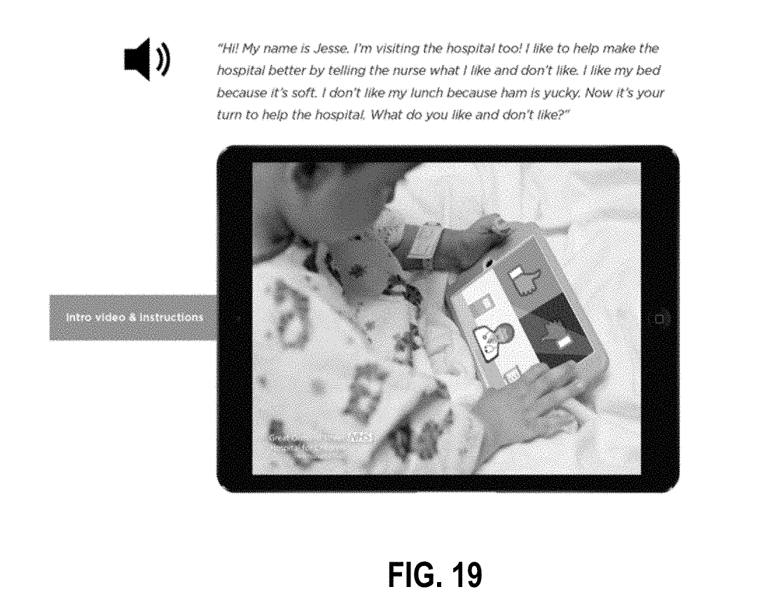

FIG. 19 is an example of a patient interacting with form interface. A young patient is using the form interface to provide feedback on a physician, the food, the bed and other features using visual elements. The patient can also submit audio feedback.

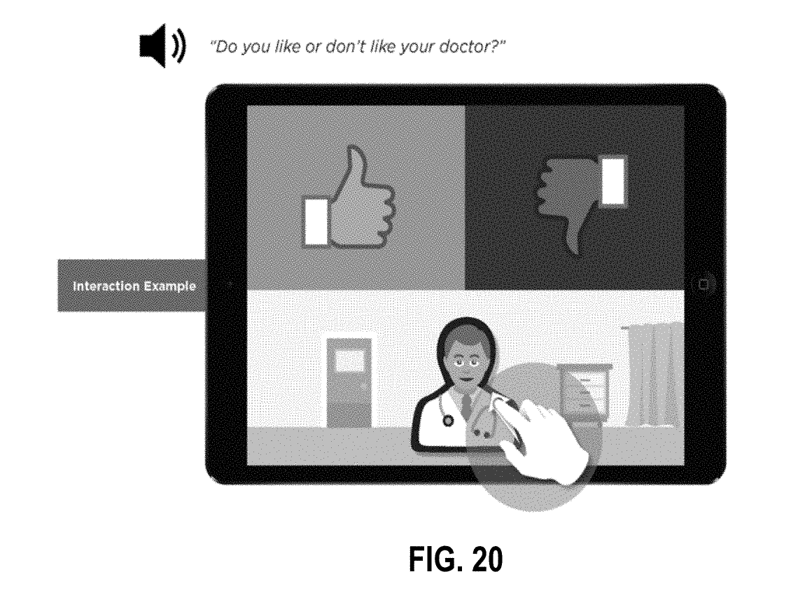

FIG. 20 is an example form interface with a feedback feature according to some embodiments. The form interface includes a visual element for a thumbs up or like and a visual element for a thumbs down or just like. The form interface includes a visual element representing a physician. The form interface can be used to provide feedback relating to the physician using the visual elements.

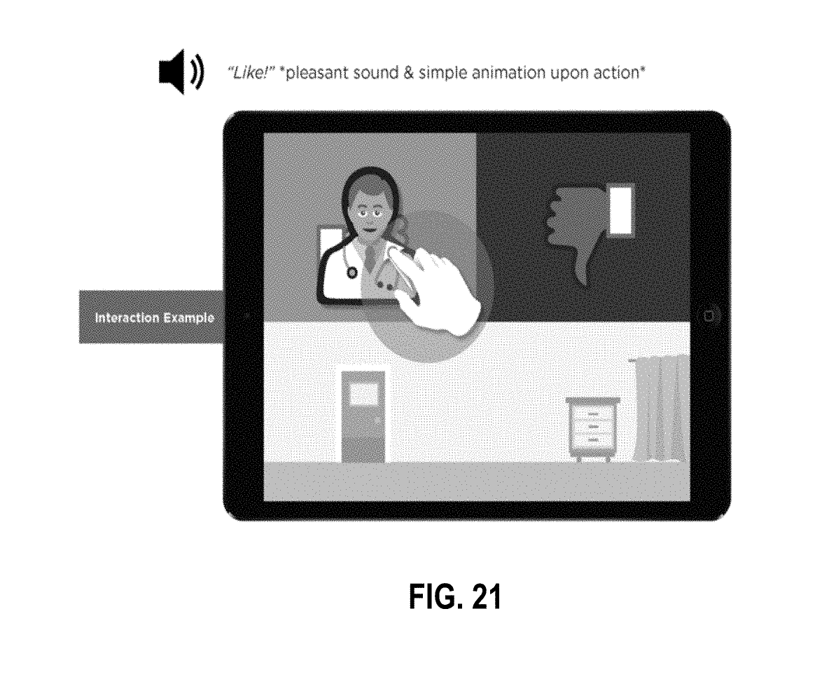

FIG. 21 is an example form interface with a feedback feature according to some embodiments. In this example, a patient interacts with the form interface to drag the visual element representing the physician to the visual element for like to provide feedback that the patient likes the physician. The collected feedback can be stored automatically to populate field values associated with feedback for the physician.

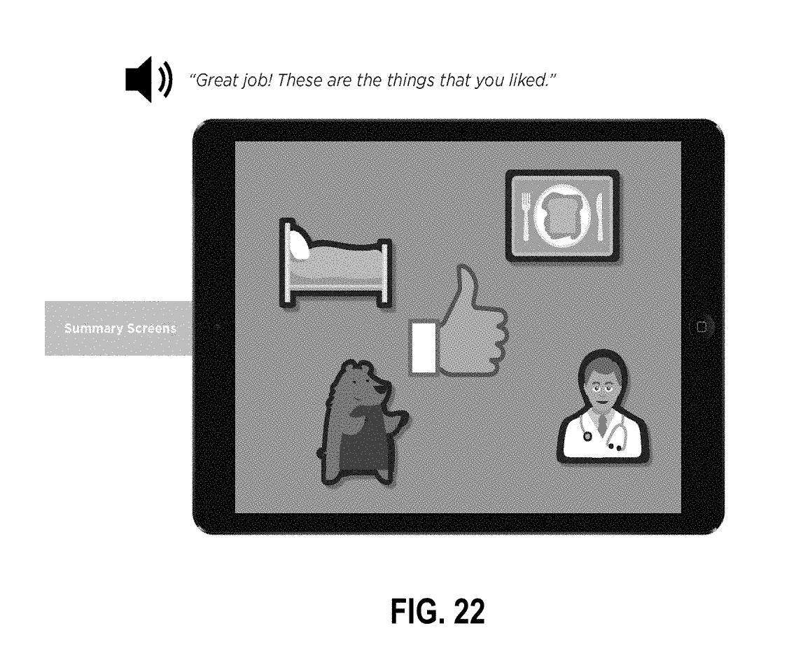

FIG. 22 is an example form interface with a feedback feature according to some embodiments. In this example, the form interface includes visual elements representing things that the patient liked about the health care facility experience. This can be a summary screen based on the collected feedback.

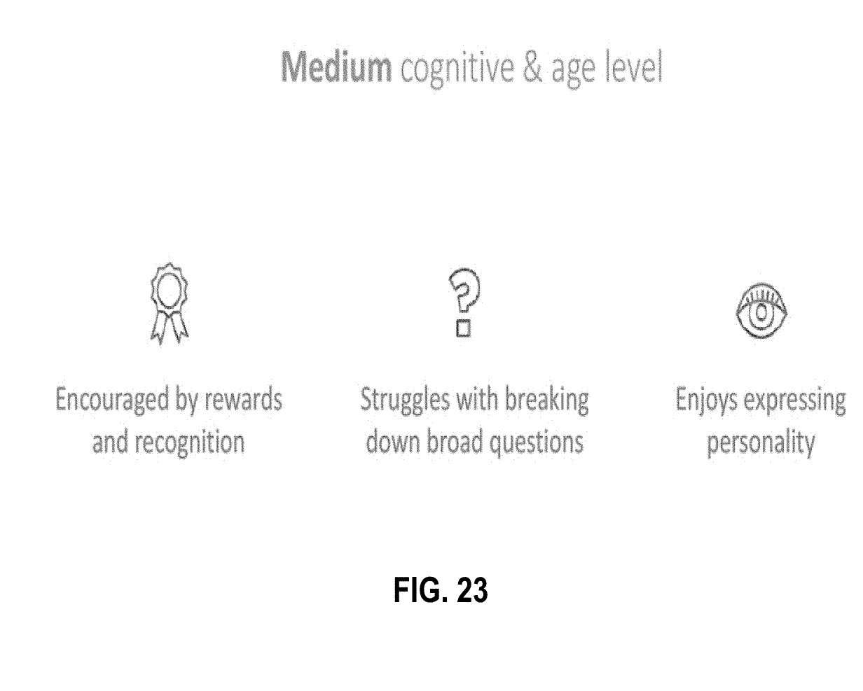

FIG. 23 is an example list of factors for people with medium cognitive and age level. People with medium cognitive and age level can be encouraged by rewards and recognition, can struggle with breaking down broad questions, and can enjoy expressing personality. These factors can be used to generate a dynamic form interface suitable for a person with medium cognitive and age level.

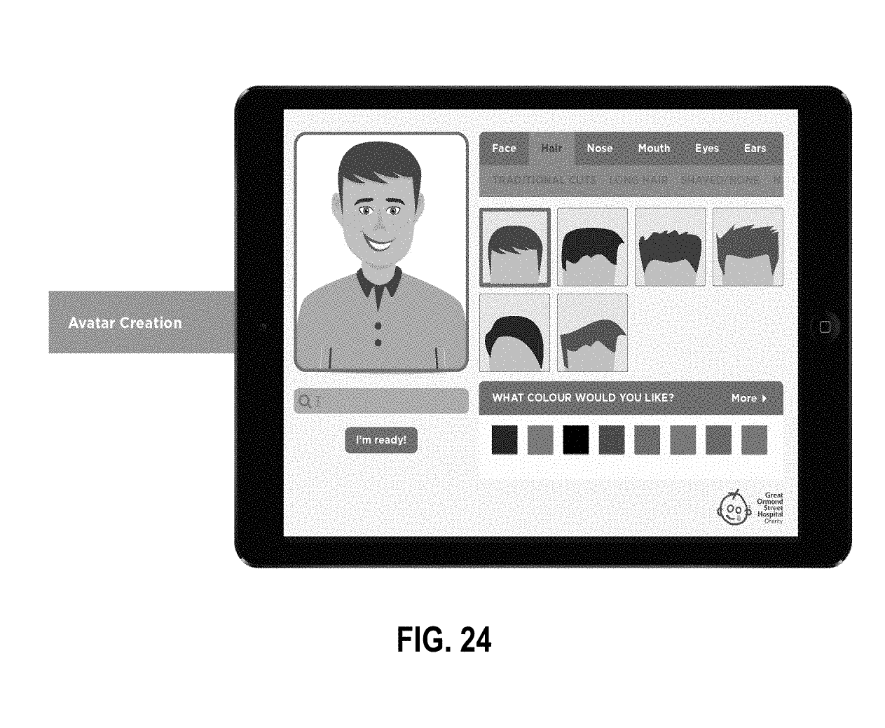

FIG. 24 is an example form interface with an avatar creation feature according to some embodiments. The form interface can include visual elements representing an avatar along with features for the avatar such as face, hair, nose, mouth, eyes, and years. This enables a patient to generate a virtual representation that they are comfortable or associate with.

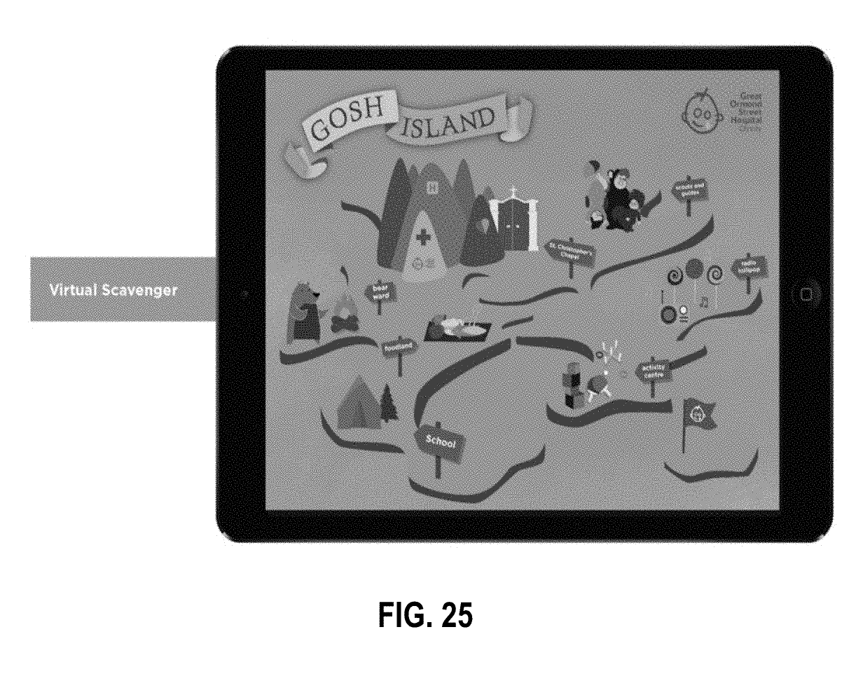

FIG. 25 is an example form interface according to some embodiments. In this example, the form interface provides a game that can facilitate the collection of relevant data.

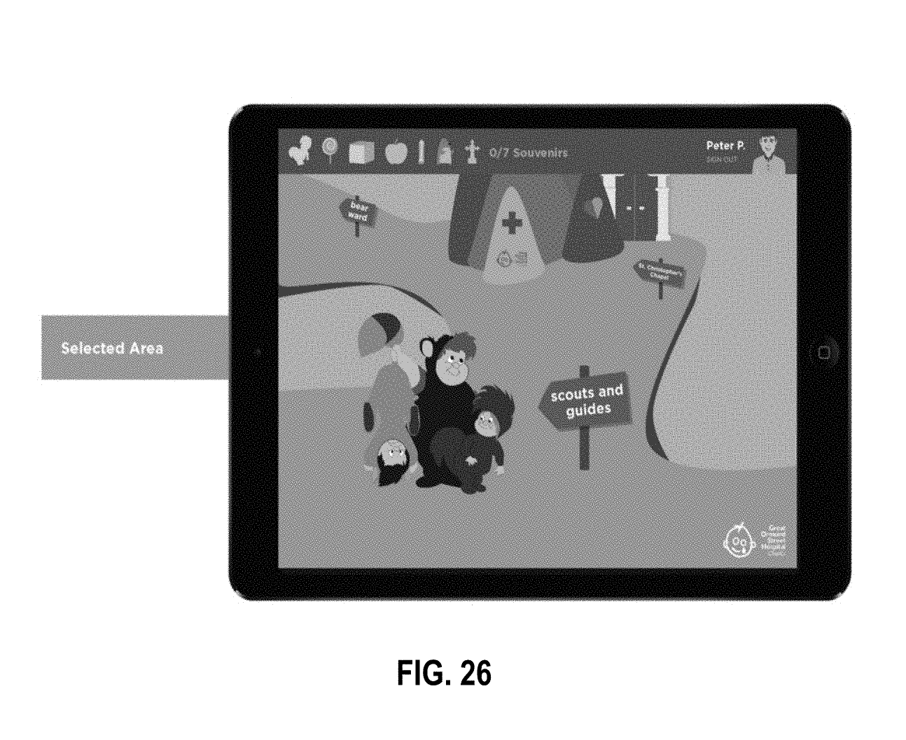

FIG. 26 is an example form interface according to some embodiments. In this example, the form interface provides a game to collect souvenirs.

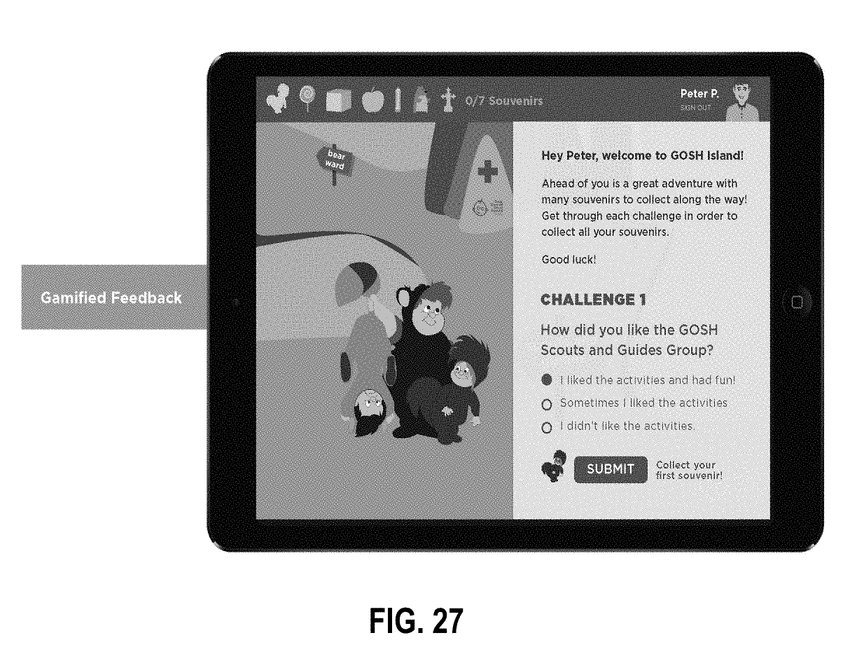

FIG. 27 is an example form interface with a challenge feature according to some embodiments. In this example, the form interface provides a challenge to engage the user.

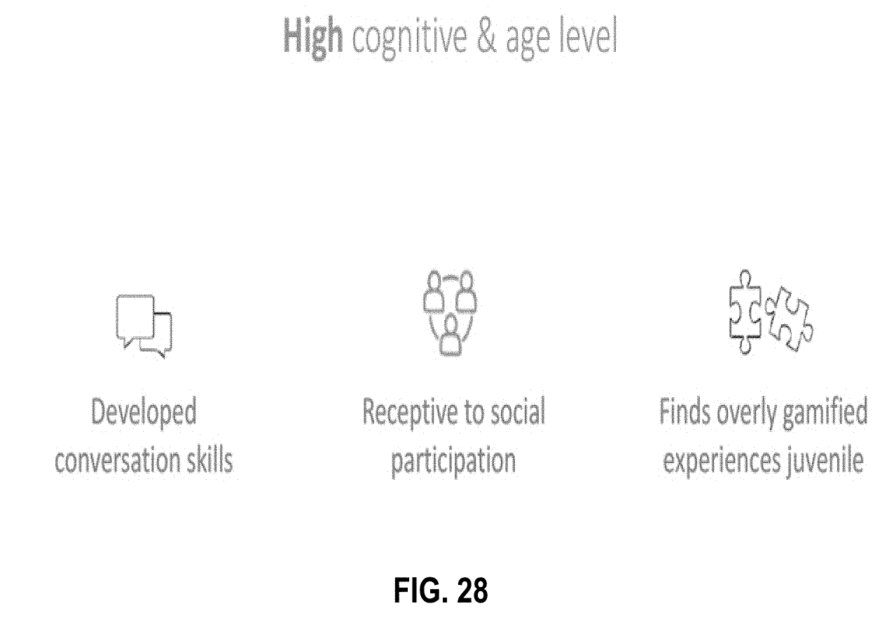

FIG. 28 is an example list of factors for people with high cognitive and age level. People with high cognitive and age level can develop conversation skills, can be receptive to social participation, and may overly game of find experiences juvenile.

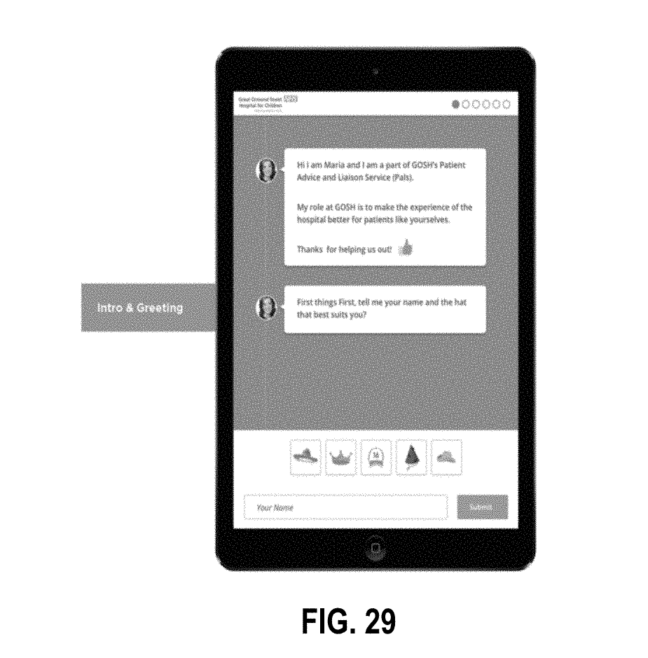

FIG. 29 is an example form interface with a greeting feature according to some embodiments. In this example, the form interface includes a messaging and greeting feature. The greeting feature may include a question. The message feature includes the ability to drag different visual elements to represent healthcare data and responses.

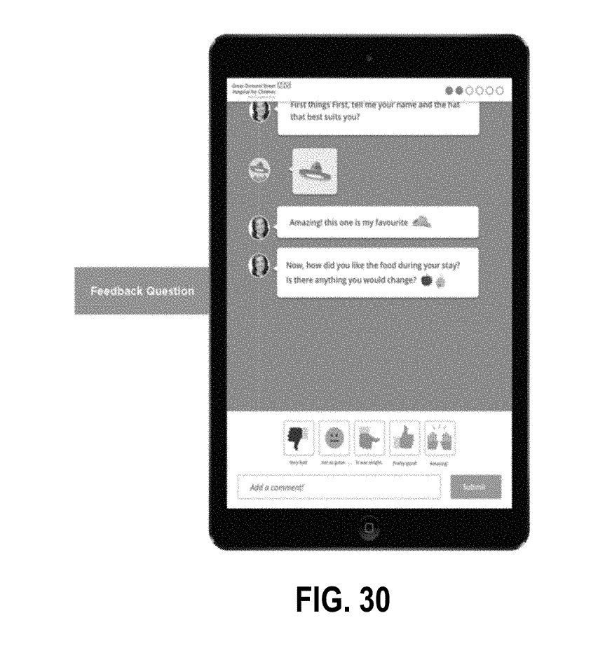

FIG. 30 is an example form interface with a feedback feature according to some embodiments. In this example, the form interface includes a messaging and feedback feature. The message can include a question. The message feature includes the ability to drag different visual elements to represent feedback data.

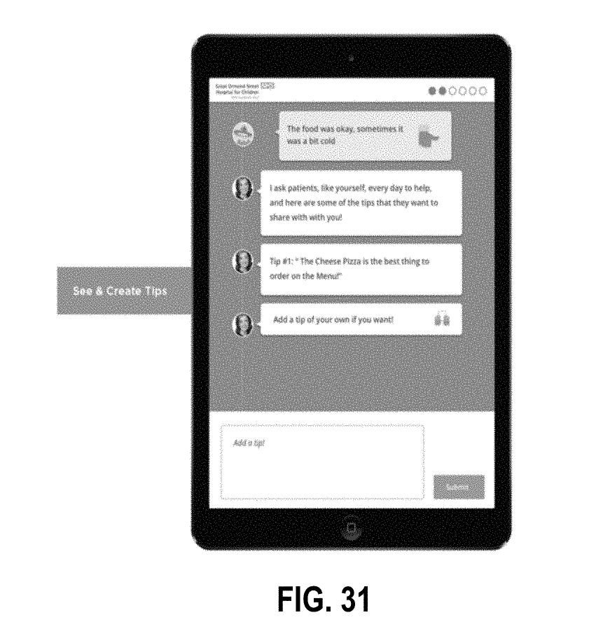

FIG. 31 is an example form interface of the messaging feature according to some embodiments. In this example, the form interface includes a messaging feature that enables a healthcare provider to give tips to improve the health care experience. This may increase the amount of positive feedback received. The tips can be automatically generated based on collected feedback. For example, collected feedback may indicate the person does not like the food and the tip can suggest an item to try on the menu.

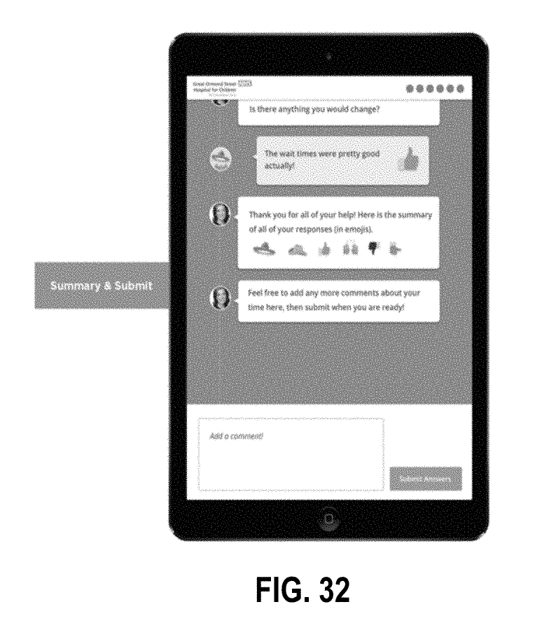

FIG. 32 is an example form interface with a messaging feature according some embodiments. In this example, the form interface includes a comment feature and a report feature to summarize the collected feedback.

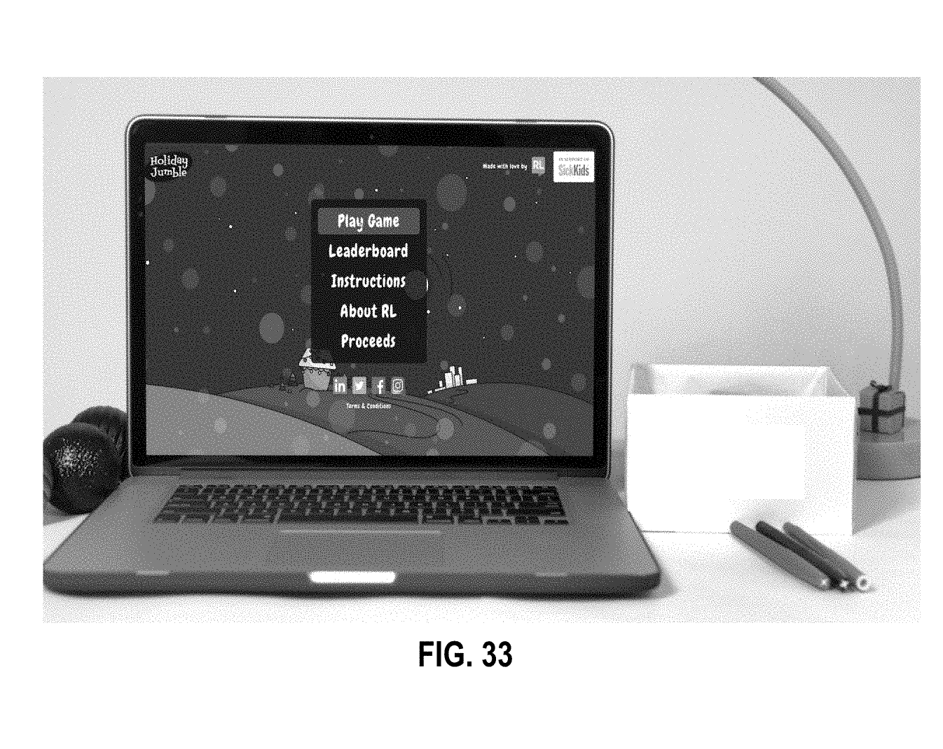

FIG. 33 is an example form interface according to some embodiments. In this example, the form interface provides options to a patient including playing a game to collect data regarding the healthcare experience.

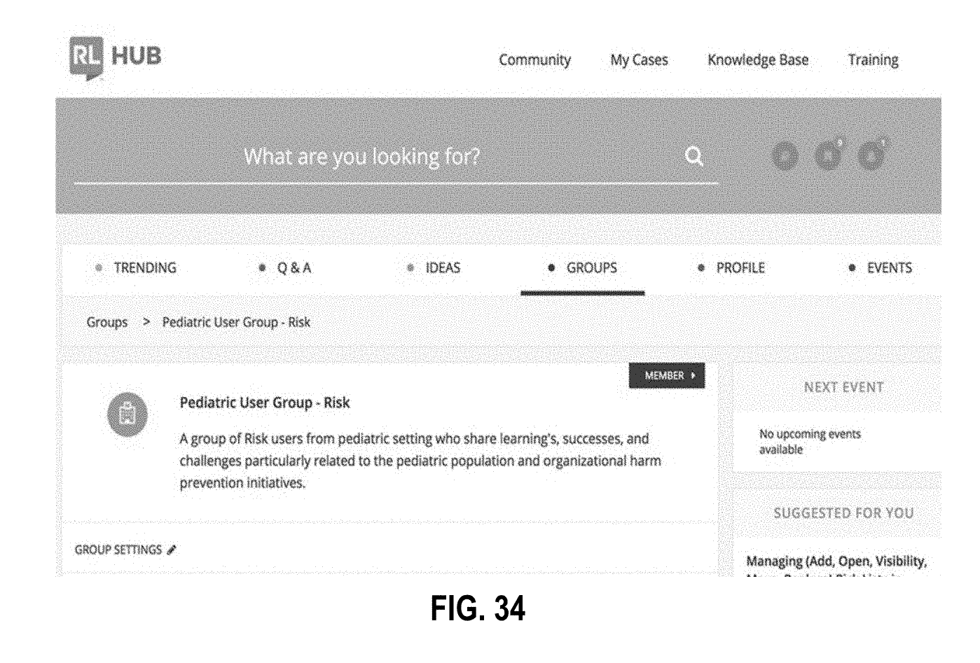

FIG. 34 is an example form interface according to some embodiments. In this example, the form interface includes a patient hub to find further information.

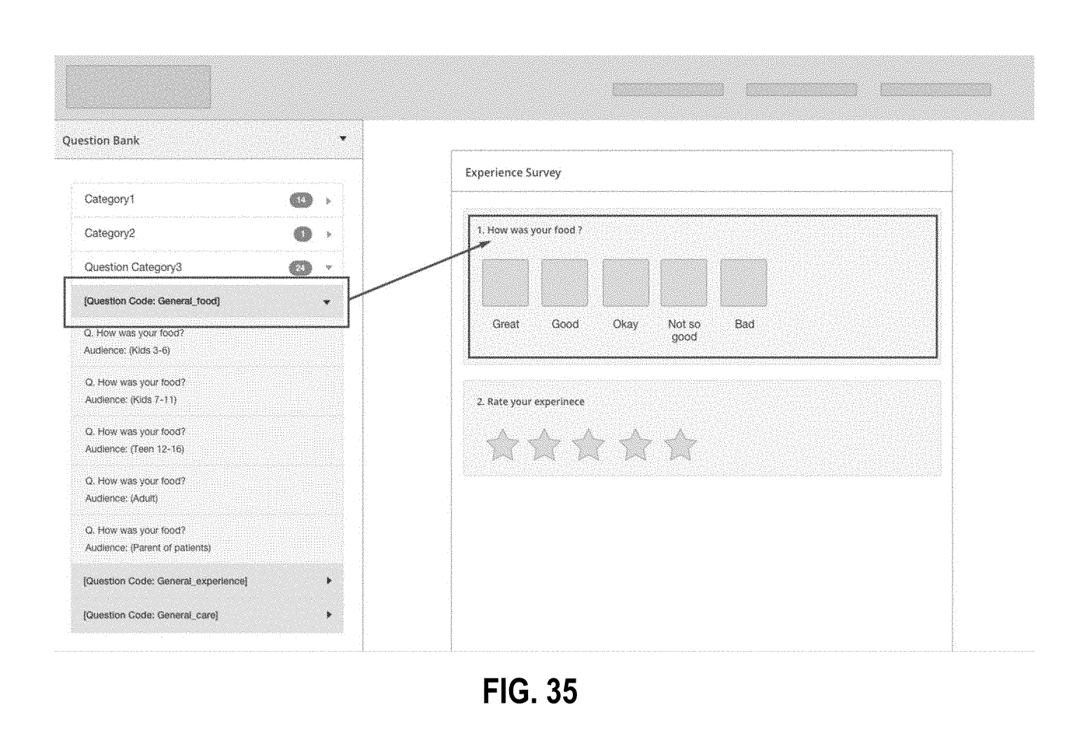

FIG. 35 is an example form interface for an administrator form interface, according to some embodiments. In this example, the form interface allows for the generation of a question generally directed to the quality of food.

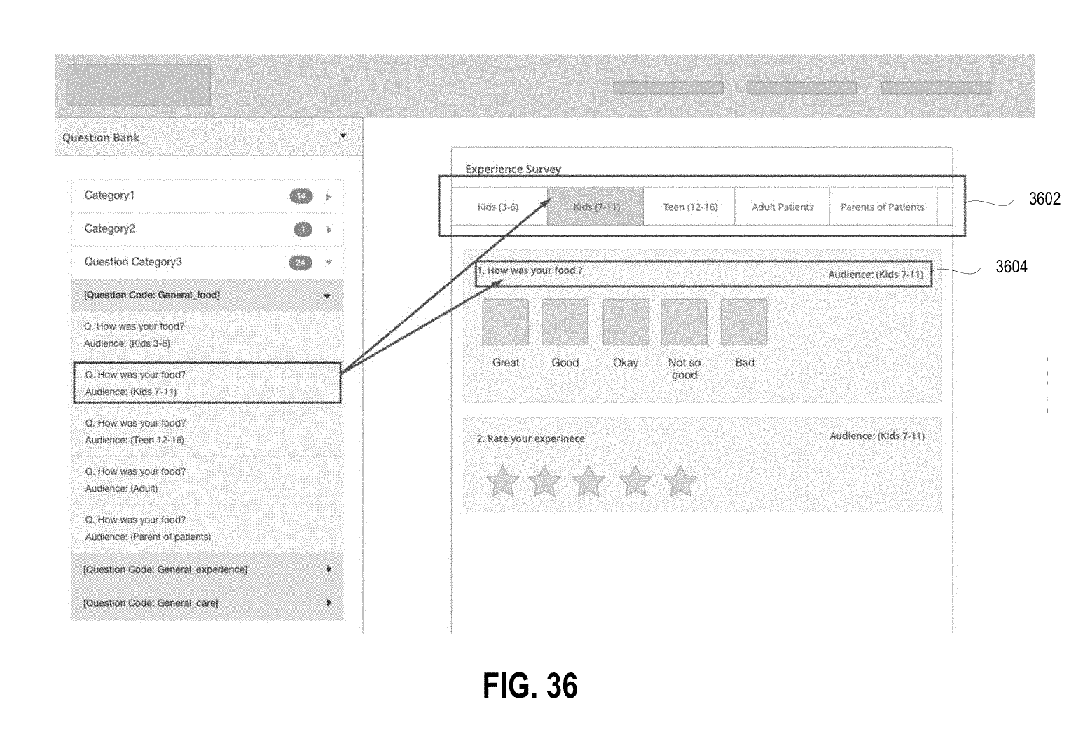

FIG. 36 is an example form interface for an administrator form interface, according to some embodiments. In this example, the form interface allows for the generation of variations of a question related to food, shown in the widget bar 3602, which controls the visual elements displayed in the question. The specific context for the question directed to a “kids” cognitive level (in this case, age), is shown at 3604.

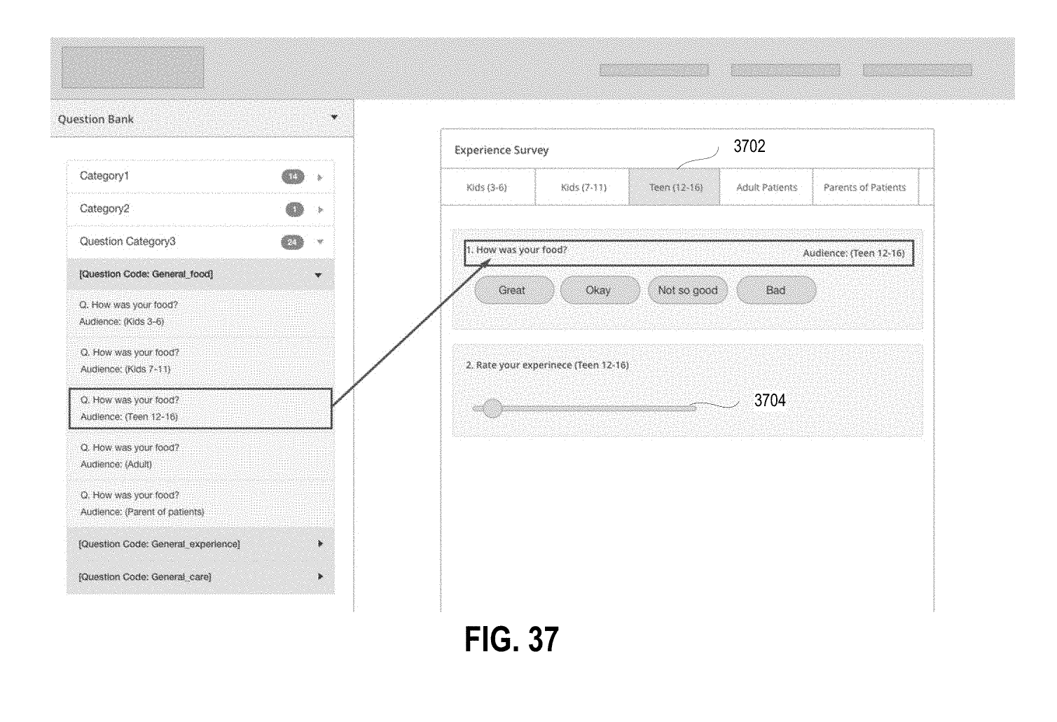

FIG. 37 is an example form interface for an administrator form interface, according to some embodiments. In this example, the form interface allows for the generation of variations of a question related to food, shown in the widget bar 3702, which controls the visual elements displayed in the question. The specific context for the question directed to a “teen” cognitive level (in this case, age). An additional visual element, the slider bar, 3704, is rendered for those in the “teen” cognitive level.

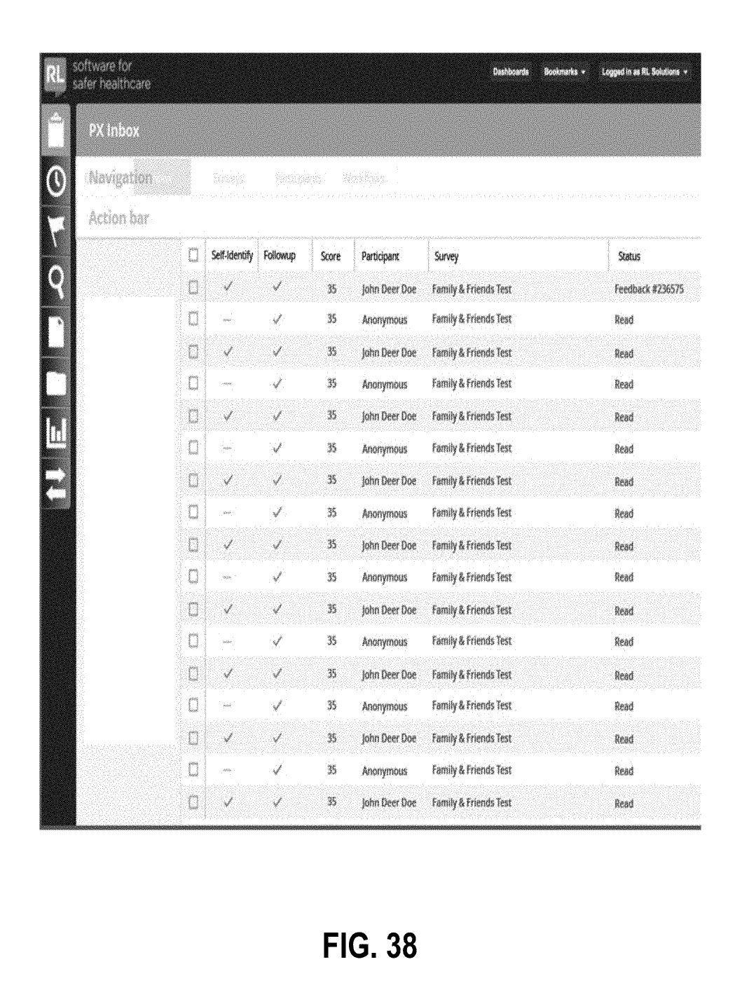

FIG. 38 is an example administrator interface for tracking distribution of a survey, according to some embodiments.

The various examples of form interfaces described and shown include different types of visual elements to facilitate the collection of relevant data. The visual elements are mapped to different form fields to automatically populate corresponding field values in data storage 202. Healthcare data can include feedback data and other data collected from a patient or healthcare system.

The survey numbers are shown as an example, and the survey score may indicate for example, a score assigned by a patient experience officer to a survey based, for example, on whether the survey was requested by the user, was pushed on the user (e.g., follow up), and whether any privacy options are requested (e.g., anonymous basis). Further, a status flag may be assigned indicating that there is a status associated with a response (e.g., read, flagged).

FIGS. 39A and 39B are data model diagrams illustrating the improved backend table relationships that support the improved metabase of some embodiments. FIGS. 39A and 39B are meant to be read side-by-side as FIG. 39A continues horizontally across to FIG. 39B.

In FIGS. 39A and 39B, a series of meta_tables 3904, meta_lists 3906, and meta_list_items 3908 are shown, in conjunction with the meta_fields 3902. These are utilized to configure the metabase such that the variety of questions, stored in the question_library,

Question_type 3924, question_semantic code 3926, can be adapted into an existing taxonomy such that an expanded taxonomy is supported by way of the customized metabase.

For applications configured for interoperation with a fixed schema or data structure, the metabase configured accordingly allows for the improved adoption of the question variations (and their associated customized automatic rendering of variations of visual controls, form elements, and interface elements) on these applications without further modification of the applications and their corresponding interfaces.

In operation, a patient experience officer can first define a ‘patient group’, or a group of patients that are of specific interest to a survey ‘campaign’ at the organization using administrator device 104. An example could be ‘patients under the age of 18 who have been discharged from the hospital.

Patient groups can be defined be creating an EXPRESSION using the RLQL query language. The query language (in complimenting patent application), uses natural language that allows users to express a query or a filter on a set of data, in this case patients.

In the example below, there are patients with type 2 diabetes, and patients with peanut allergies. The corresponding criteria in the context of the domain-specific language is provided below.

| PK_ID | DISPLAY_NAME | CRITERIA |
|---|---|---|
| 1 | Patients with Type 2 | <RLQL>Any living Patient with condition |
| | Diabetes | ‘Diabetes mellitus type 2’</RLQL> |
| 2 | Patients with peanut | <RLQL>Any living Patient with known |
| | allergies | allergy to ‘peanuts’</RLQL> |

The user can use the RLQL query language to define conditions based upon fields dealing with demographic information about the patient (e.g., age, gender, etc.), admission data (e.g., date admitted, discharged), as well as a variety of data from surgery, lab, pharmacy, etc., feeds that are captured in the system 100 using the HL7 Engine listener, or using a query on demand system to the EHR.

These fields are linked to meta-fields using the metabase, where patient information may be represented using meta-structures, such as meta-tables, meta-relationships, among others.

A patient group can be a ‘dynamic group’, meaning that it is represented only by an expression, instead of a fixed list of patients, however a static group (fixed list of patients by ID) is also an available option. For the purpose of illustration, a dynamic group is shown in the proposed model. This expression defining the group is evaluated at the time of rule execution/trigger when the survey invitation is created.

While a patient group serves the purpose of segmenting patients to receive groups of surveys when a trigger event occurs, a target audience is a different concept. A target audience refers to the definition of a group of a individuals characterized by a select group of parameters: age group, recognition level, communication level, mental capacity level, and physical ability. A patient experience officer can configure multiple audiences based on a variety of combinations between these parameters.

The purpose of a target audience is to be able to link this concept to questions variations that are applicable or appropriate for those target audiences, which in turn may be rendered via different control types that allow for different interaction mechanisms and paradigms of interaction (e.g., through appropriate visual elements).

| PK_ID | DISPLAY_NAME | ENTITY | AGE_GROUP |
|---|---|---|---|
| 1 | Young Kids | Patient | Kids 3-6 |
| 2 | Kids | Patient | Kids 7-11 |
| 3 | Teens | Patient | Teen 12-16 |
| 5 | Adult Patients | Patient | Adult |
| 6 | Parents of Patients | Parent | Adult |

The target audience can be provided in the form of a table that allows various characteristics to be ‘mapped together’. This relationship is not necessarily 1 to 1. Many patient groups that target different recipients may be mapped to a single target audience (1 to n). A simple example is 2 patients groups that are “teens with diabetes”, and ‘teens without diabetes”. while these target 2 populations of patients in a hospital, they may be both mapped to a target audience of “teenager”, a target audience created by a patient experience officer to render the survey using controls and interactions appropriate for teenagers.

| | | FK_TAR- | |
|---|---|---|---|
| | FK_PA- | GET_AU- | |
| PK_ID | TIENT_GROUP_ID | DIENCE_ID | RULE |
| 1 | Patients with Type 2 | Young Kids | <RLQL>patient age |
| | Diabetes | | group between 3 and |
| | | | 6</RLQL> |
| 2 | Patients with Type 2 | Kids | <RLQL>patient age |
| | Diabetes | | group between 7 and |
| | | | 11</RLQL> |
| 3 | Patients with Type 2 | Teens | <RLQL>patient age |
| | Diabetes | | group between 12 and |
| | | | 16</RLQL> |
| 4 | Patients with Type 2 | Adult Patients | <RLQL>patient age |
| | Diabetes | | group greater than |
| | | | 16</RLQL> |
| 5 | Patients with peanut | Young Kids | <RLQL>patient age |
| | allergies | | group between 3 and |
| | | | 6</RLQL> |
| 6 | Patients with peanut | Kids | <RLQL>patient age |
| | allergies | | group between 7 and |
| | | | 11</RLQL> |
| 7 | Patients with peanut | Teens | <RLQL>patient age |
| | allergies | | group between 12 and |
| | | | 16</RLQL> |
| 8 | Patients with peanut | Adult Patients | <RLQL>patient age |
| | allergies | | group greater than |
| | | | 16</RLQL> |

A survey is made up of multiple questions that a recipient can respond to. In many cases, a survey that contains a specific set of questions is meant to be sent to a variety of patients that all have different ages, cognitive abilities, and physical abilities.

| PK_ID | QUESTION_TYPE |
|---|---|
| LIST | Picklist |
| RATING | Rating |
| MEMO | Multi-line Text |

The innovative healthcare system 100 is then utilized to create a single survey with a set of questions, and have that survey be rendered in different ways based upon the target audience (see definition above) of the recipient individuals.

The survey may be displayed differently, contains different controls to represent response fields, and can contain different styling and presentation aspects depending on the target audience.

| PK_ID | QUESTION_TYPE |
|---|---|
| DROPDOWN | LIST |
| TILE | LIST |
| BADGE | LIST |
| WHEEL | LIST |
| SLIDER | RATING |
| IMAGE_SELECT | RATING |
| HTML_EDITOR | MEMO |

A fundamental linkage here exists in the creation of “questions”, the key building block of the survey. The table below indicates where some questions are mandatory or not, and associated question identifiers and survey identifiers.

| | | | IS_MAN- |
|---|---|---|---|
| PK_ID | FK_QUESTION_ID | FK_SURVEY_ID | DATORY |
| 1 | 1 | 1 | TRUE |
| 2 | 2 | 1 | TRUE |
| 3 | 3 | 1 | TRUE |
| 4 | 4 | 1 | TRUE |
| 5 | 5 | 1 | TRUE |
| 6 | 6 | 1 | TRUE |

Questions are part of a question library 3922, and can store strings of text to display to the user, and visual element selections, among others, but also contain linkages (foreign keys) to question type 3924, semantic code 3926, list id (for lists), as well as control type 3920, and target audience 3918.

These linkages are all described below:

Question 3922—Target Audience 3918->A question record 3922 is meant for a target audience. There may be multiple instances of a question with the same purpose (e.g., to determine how the food at a hospital was), but there is a separate data row for each question.

Question 3922—Semantic Code 3926->This semantic code 3926 is an identifier for the question that links it to a common data point for reporting and statistics. For example, if the survey is asking a fundamental question about the quality of the food, the system 100 assigns a semantic code 3926 to that question intent. There may then be multiple questions linking back to that same data point for reporting, but those questions may be all tailored toward different audiences.

Question 3922—Question Type 3924->This linkage allows the system to set a ‘data type’ for a question 3922. For example, this could be an Integer value, or a string value. This linkage is important, as all questions linked to the same semantic code 3926, in some embodiments, must also follow the same data type for consistent reporting.

Question 3922—Control Type 3920->This denotes the control type 3920 that is used to render that question 3922. There can be many control types 3920 for each question type 3924 or data response type. For example, two controls that represent different styled sliders can still provide an integer result value that conforms to the same data type. The system 100 can have different questions, all of the same data type, but just rendered using different controls 3920, and potentially linked back to the same semantic code 3926.

| | | FK_QUES- | FK_TARGET_AUDI- | | | |
|---|---|---|---|---|---|---|
| PK_ID | QUESTION_TEXT | TION_TYPE | ENCE | FK_SEMANTIC_CODE | CONTROL_TYPE | FK_LIST_ID |
| 1 | Was your food yummy? | LIST | 2 | food | TILE | 100001 |
| 2 | Rate your overall | RATING | 2 | experience | IMAGE_SELECT | 100002 |
| | experience | | | | | |
| 3 | Were you involved in | LIST | 2 | care | TILE | 100003 |
| | decisions about your | | | | | |
| | care and treatment? | | | | | |
| 4 | How was your child's | LIST | 5 | food | BADGE | 100001 |
| | food? | | | | | |
| 5 | Rate your overall | RATING | 5 | experience | SLIDER | 100002 |
| | experience | | | | | |
| 6 | Were you involved in | LIST | 5 | care | BADGE | 100003 |
| | decisions about your | | | | | |
| | child's care and | | | | | |
| | treatment? | | | | | |

All of these linkages together form the concept of questions of a survey that can share a common question type 3924 and semantic code 3926 (e.g., the ‘same’ fundamental question being asked to the recipient), but using different control types 3920 and an association of each question to a target audience 3916, this model forms a ‘variation’ concept on a survey question that allows the questions to be included in a survey definition, and then at the time of invitation, depending on the recipient type's grouping as belonging to a target audience, a different question row may be selected to solicit that information or response from the end user, all while linking back to a same semantic code or data point for normalized reporting.

A survey 3919 is defined as a definition in the SURVEY table with properties of that survey. A SURVEY_QUESTION table 3928 holds which individual questions may be included within that survey, as well as custom logic on that set of questions (per row).

In some embodiments, it is important to separate the modelling for the UI/Presentation layer, as the user may or may not be adding each specific question into the survey definition based on that target audience, but may simple add a ‘semantic’ code 3926 from the presentation layer, which then translates into rendering one or more survey question records linked to the keys in the question library that correspond to the available target audiences.

As well, there may be a case where there is only a single rendering of a question for a semantic code, as this question may be only applicable to a single audience. This also demonstrates a valid case. For example, there may be a case where there are 3 questions on a survey that are shared or needed to be answered by all audiences, however there may be questions that are ONLY applicable to a subset of those audiences, and that question will still be included in the survey definition, but not rendered when a member of that excluded audience is answering the survey 3919.

The metabase linkage to questions 3922 is also shown in the data model in that list items from questions option sets are linked to lists and lists items that are used in the metabase.

The invitation to a survey is generated when a specific event trigger occurs on a patient that belongs to a defined patient group. A simple example of this trigger could be a discharge. When the trigger occurs, the system 100 then computes and generates and invitation to be added to the Invitation table 3921.

An invitation 3921 has many attributes, specifically including a Survey and Participant ID, but also importantly including which TARGET AUDIENCE the patient belongs to. When the survey is being rendered, the invitation 3921 is referenced and then based on the associated target audience of the participant, specific questions in the SurveyQuestions that match that target audience will be displayed and shown to that recipient prior to providing access to applications, a local network, and network resources, for example.

In an operational example, rule based mapping can be provided whereby a patient experience department of a hospital seeks to benchmark how patients with specific medical conditions rate the food during their stay the hospital.

In this example, a metabase is maintained wherein there is a dictionary of field objects stored in association with a particular schema or set of fields. As variations are generated for questions that are present in the schema or set of fields, the metabase is configured to be adaptive to support the additional variations by dynamically re-mapping and re-purposing fields (e.g., lesser used or unused fields). The metabase stores such linkages (meta-relations) within meta-tables 3904 and meta-lists 3906, which are then connected to meta-fields 3902 across linkages 3910 and 3912.

The meta-fields 3902 are an expandable set of fields (e.g., with a dynamic number of columns) that allow for repurposing of existing fields to provide a flexible metaschema that allows a new taxonomy to be accommodated and adopted without modifications to applications that depend on the metabase. Form fields may have parent-child relationships with one another, and are utilized to track variations on a question that may be posed, as well as the logical condition as to when a particular question should be posed (e.g., responsive to the estimated cognitive level).

As variations in questions are generated in question library 3922, their mappings to target audience 3918 representing logical conditions upon which they will be invoked, additional field objects are added to the metabase by way of meta-tables 3904 and meta-fields 3902. Meta-tables 3904 may correspond to physical database tables, field objects may correspond to physical columns in a database table, and meta-relations may correspond to foreign keys used to reference field objects and meta-tables.

Metadata may store additional attributes and relationships in additional metabase tables 3904. For example, metadata may use primary and foreign keys to link attributes relating to a field object to meta-lists 3906 and meta-list items 3908. Meta-list 3908 may store potential field values for a field object. Meta-list items 3908 may store attributes pertaining to a potential field value. For example, a meta-list item may store sequence information for a potential field value within the meta-list 3906.

Where the question variations require expansion of the metabase columns, the form engine 204 is configured to establish one or more new columns corresponding to the one or more additional user-defined field objects.

For the one or more additional user-defined field objects, new meta-relations are established defining the one or more additional user-defined field objects as new child nodes to corresponding parent nodes of the one or more additional user-defined field objects, so that variations and linkages can be indicated. These linkages, for example, may be between questions of the question library 3922, and may include linkages to different control types 3920, question types 3924, semantic code 3926, among others.

When a form is generated, the application invokes the form engine 202 and renders form elements in accordance with visual controls and graphical user elements returned by form engine 202. The application may provide a naive request to the form engine 202, for example, indicating that a particular survey or form needs to be rendered and requests rendering commands and control sets.

The form engine 202, in generating the electronic form, calls the metabase to retrieve information including the corresponding meta-field 3902. Based on a traversal of the meta-linkages and information stored on metatables 3904, meta-lists 3906, and meta-list items 3908 in view of the estimated cognitive level of the individual being targeted, the form engine 202 is able to selectively render a set of control types 3920 appropriate for the question variation being posed from question library 3922. From the perspective of the application, responsive to a request for a survey, the application may invoke control the presentation of a form or survey in accordance with its regular fixed schema and/or database calls.

However, the metabase mechanism is used as an intermediary that adaptively re-configure the actual presentation of the form or survey in accordance with the expanded taxonomy of the variations, and the metabase traversable to identify cognitive-level appropriate rendering controls. Accordingly, an improved electronic form is rendered on user device 102 that includes an ordered collection of form fields instantiated using the field objects, the ordered collection based at least on the relationships stored in the metabase including at least the meta-tables, the meta-relations, and the new meta-relations, the ordered collection of form fields being a set of visual elements. The improved electronic form is adapted for contextual estimations of the individual's cognitive level.

In particular, the system 100 control the display of user device 102 to automatically return and display, from the system interface to the user interface component 210 via the network 106, the set of visual elements obtained from the metabase. Each visual element corresponds to a potential field value associated with the corresponding field object used to instantiate a corresponding form field responsive to the estimated cognitive level of the individual selected from a range or discrete set of cognitive levels.

The form interface 210 can be utilized to receive selections or other input (e.g., answers) from the individual, and similarly, store these selections either in a raw format or a normalized format in data storage 202. In some embodiments, metadata tags, storing metabase linkages, are attached to the response inputs so that a downstream analyst or reporting mechanism is able to re-generate or re-trace the visual interface customizations that occurred as a result of the question variations, for example, to track potential bias due to modified presentment, among others.

As an example, an administrator would configure 2 patient groups, one targeting “Patients with Type 2 Diabetes” (PK_ID 1 in the sample data) and another “patients with peanut allergies” (PK_ID 2) using a domain-specific-language (e.g., RLQL). This criteria will be evaluated against a dataset which is sourced from HL7 feeds and return a list of matching patient records.

On the form engine 204, the question would need to be displayed with different control types to engage people of different age groups and cognitive abilities. The survey questions 3922 are related via a foreign key to a table TARGET_AUDIENCE 3918 which can, for example, be defined based on AGE_GROUP (e.g. child vs. teen vs. adult) and ENTITY to denote the type of participant in the survey (e.g. patient vs. parent). Other types of definitions are possible, such as through estimated cognitive level. In some embodiments, the estimated cognitive level is based primarily on age as provided in electronic health records, and adjusted accordingly based on other known information (e.g., level of education, postal code, procedural history, treatment history, therapeutic drug administration history).

For example, a simpler variation of questions may be administered to those who have recently completed a course of dialysis or chemotherapy. These individuals may be exhausted or tired, and simpler questions and visual elements may be necessary to elicit a useful survey response.

The system 100 is configured to map records from a specific patient group to their appropriate target audience in the survey domain. In an example embodiment, this mapping is conducted using a rule-based approach targeting additional attributes of the patient records and narrowing down the appropriate target audience based on their age.

For example, a simple criteria will indicate that “patients with current age of 3-5” will map to target audience “Young Kids” in the survey. This will result in the survey rendering the appropriate wording and control type at the time of execution. This relationship is represented in the GROUP_TO_TARGET_AUDIENCE_MAPPING 3916 table in the database.

Each question in the QUESTION_LIBRARY table 3922 is assigned a QUESTION_TYPE 3924. Each QUESTION_TYPE 3924 can have multiple records in CONTROL_TYPE 3920 to indicate that the same type of data can entered via a variety of front-end controls. The system includes a mapping to recommend a CONTROL_TYPE 3920 based on the age group of the selected target audience.

To represent relationship between different questions, the system of some embodiment groups different questions using a “QUESTION_SEMANTIC_CODE” 3926 reference table, enabling an administrator to relate different questions together before a survey is sent out to users which will remove the need for a data sanitization process commonly required after results are received.

In the example set shown in the tables provided earlier in this application, the tables illustrate that both questions with PK_ID 1 and 4 have the same code of “food” to denote that are asking about the same thing with different wording. This will enable a normalized dataset for reporting downstream and more accurate analysis of the results.

The improved form engine 204 invokes the metabase for use in several areas, including

The set of possible answers that a question of type “LIST” can have is stored in the TBL_META_LISTS 3906 and TBL_META_LIST_ITEMS 3908 tables. For example, a question with possible answers (“Yes”, “No”, “Maybe”) is referenced as a list with each answer represented as a list item. This is implemented using a nullable foreign key in the QUESTION_LIBRARY table 3922.

Attributes referenced in the RLQL condition set are rows in the TBL_META_FIELDS 3902 table which extends the model from the physical tables storing the data and the HL7 feeds that populate them. The RLQL query itself can be stored in a document based format using an XML datatype column.

The ENTITY attribute of the target audience (used to separate “parent” vs “patient”) is a reference to TBL_META_TABLES 3902 which can extend lower level implementation of how the data is physically represented. For example, a parent can be represented in the persistence layer as Related Person where relationship type=“parent” while in the meta layer it can be referenced directly as parent.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

One should appreciate that the systems and methods described herein may provide example technical effects and solutions using different representations of data for e.g. better memory usage, improved processing, improved bandwidth usage, improved usability.

Various example embodiments are described herein. Although each embodiment represents a single combination of inventive elements, all possible combinations of the disclosed elements include the inventive subject matter. Thus if one embodiment comprises elements A, B, and C, and a second embodiment comprises elements B and D, then the inventive subject matter is also considered to include other remaining combinations of A, B, C, or D, even if not explicitly disclosed.

The term “connected” or “coupled to” may include both direct coupling (in which two elements that are coupled to each other contact each other) and indirect coupling (in which at least one additional element is located between the two elements).

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information.

Although the embodiments have been described in detail, it should be understood that various changes, substitutions and alterations can be made herein without departing from the scope as defined by the appended claims.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

As can be understood, the embodiments described above and illustrated are intended to be examples only.

What is claimed is:

1. A computer system for dynamically generating an electronic form for receiving electronic data relating to a health care event, the electronic form adapted to provide a flexible meta-schema allowing an expanded taxonomy to be adopted or accommodated without modifications to applications that process the electronic form, the system comprising:
a user interface component on a connected device configured to receive a request to create the form;
a storage device having a metabase associated therewith to maintain the meta-schema; and
at least one processor configured to execute instructions to provide a form engine and a system interface, the form engine configured to:
receive a data set representative of one or more electronic health records associated with an individual;
determine an estimated cognitive level of the individual by parsing the data set;
provide a healthcare event management system including a storage device and a processor, the storage device having a metabase associated therewith to maintain the meta-schema and the processor configured to provide a form engine and a system interface;
establish a communication link between the system interface and the user interface component over a network;
receive, at the system interface from the user interface component via the network, a request to generate the electronic form using the form engine;
store, in the storage device, a dictionary of field objects in the metabase associated with the storage device of the healthcare event management system, wherein an instance of a field object is a form field, and wherein the form field is configured to receive a field value, the metabase structuring the dictionary of field objects into corresponding meta-tables and establishing meta-relations defining parent-child relationships between nodes representing the one or more field objects of the metabase, the meta-relations being maintained as corresponding foreign keys used to reference the corresponding field objects and the corresponding meta-tables, the metabase configured to have a dynamic number of columns that is dynamic according to a number of field objects in the dictionary;
add one or more additional user-defined field objects in the metabase to accommodate an expanded taxonomy capturing field object variations representative of variations of field objects that correspond to a plurality of cognitive levels;
automatically maintain, on the metabase, electronic representations of relationships between the field objects and the form fields, the one or more additional user-defined field objects representing the expanded taxonomy, the automatic maintaining including:
establishing, by the metabase, one or more new columns corresponding to the one or more additional user-defined field objects; and
establish new meta-relations defining the one or more additional user-defined field objects as new child nodes to corresponding parent nodes of the one or more additional user-defined field objects; generating the electronic form, using the form engine configured by the processor of the healthcare event management system, wherein the electronic form includes an ordered collection of form fields instantiated using the field objects, the ordered collection based at least on the relationships stored in the metabase including at least the meta-tables, the meta-relations, and the new meta-relations, the ordered collection of form fields being a set of visual elements;
control the electronic form using the set of visual elements based at least on the relationships stored in the metabase to adopt or accommodate the expanded taxonomy;
control the display to automatically return and display, from the system interface to the user interface component via the network, the set of visual elements, wherein each visual element corresponds to a potential field value associated with the corresponding field object used to instantiate a corresponding form field responsive to the estimated cognitive level of the individual selected from the plurality of cognitive levels; and
receive, at the system interface, a selected visual element from the user interface component via the network, the selected visual element being from the set of visual elements.

2. The computer system of claim 1, wherein the expanded taxonomy includes modifications to the set of visual elements of the electronic form.

3. The computer system of claim 2, wherein the modifications to the set of visual elements of the electronic form includes at least one of a displayed font size, an object visual factoring size, and a selected visual theme.

4. The computer system of claim 3, wherein the selected visual theme is modified through a substitution of a cascading style sheet (CSS).

5. The computer system of claim 1, wherein the data set representative of the one or more electronic health records associated with the individual is retrieved using a domain specific query language.

6. The computer system of claim 1, the form engine further configured to:
determine a group of target individuals to survey identified based on one or more common characteristics extracted from one or more electronic health records corresponding to each individual of the group of target individuals;
for each individual of the group of target individuals, generate one or more individualized electronic form using the metabase; and
control one or more user interface components, each corresponding to an individual of the group of target individuals, to render the one or more individualized electronic forms.

7. The computer system of claim 1, wherein each of the new meta-relations includes a corresponding normalization factor, and the form engine is further configured to:
receive one or more data sets representative of responses from the electronic form;
traverse the metabase to identify one or more normalization factors associated with the set of visual elements utilized in generating the form;
apply the one or more normalization factors to transform the one or more data sets into a normalized data set; and
store the normalized data set in a data storage maintaining response data sets normalized to a pre-expansion taxonomy of field objects.

8. The computer system of claim 1, wherein the electronic form comprises a set of sequential interface screens; and the form engine is further configured to:
track one or more response characteristics of the individual as the interface renders each interface screens;
responsive to the tracked one or more response characteristics, re-determine the estimated cognitive level of the individual; and
responsive to a change in the estimated cognitive level of the individual, re-generate one or more remaining interface screens of the set of sequential interface screens of the electronic form.

9. The computer system of claim 1, wherein the field object variations representative of variations of field objects that correspond to a plurality of cognitive levels include differing question strings appropriate for corresponding cognitive levels of the plurality of cognitive levels.

10. The computer system of claim 1, wherein the field object variations representative of variations of field objects that correspond to a plurality of cognitive levels include differing visual elements appropriate for corresponding cognitive levels of the plurality of cognitive levels.

11. A computer-implemented method for dynamically generating an electronic form for receiving electronic data relating to a health care event at a user interface component, the electronic form adapted to provide a flexible meta-schema allowing an expanded taxonomy to be adopted or accommodated without modifications to applications that process the electronic form, the method comprising:
receiving a data set representative of one or more electronic health records associated with an individual;
determining an estimated cognitive level of the individual by parsing the data set;
providing a healthcare event management system including a storage device and a processor, the storage device having a metabase associated therewith to maintain the meta-schema and the processor configured to provide a form engine and a system interface;
establishing a communication link between the system interface and the user interface component over a network;
receiving, at the system interface from the user interface component via the network, a request to generate the electronic form using the form engine;
providing a dictionary of field objects in the metabase associated with the storage device of the healthcare event management system, wherein an instance of a field object is a form field, and wherein the form field is configured to receive a field value, the metabase structuring the dictionary of field objects into corresponding meta-tables and establishing meta-relations defining parent-child relationships between nodes representing the one or more field objects of the metabase, the meta-relations being maintained as corresponding foreign keys used to reference the corresponding field objects and the corresponding meta-tables, the metabase configured to have a dynamic number of columns that is dynamic according to a number of field objects in the dictionary;
adding one or more additional user-defined field objects in the metabase to accommodate an expanded taxonomy capturing field object variations representative of variations of field objects that correspond to a plurality of cognitive levels;
automatically maintaining, by the metabase, electronic representations of relationships between the field objects and the form fields, the one or more additional user-defined field objects representing the expanded taxonomy, the automatic maintaining including:
establishing, by the metabase, one or more new columns corresponding to the one or more additional user-defined field objects; and
establishing new meta-relations defining the one or more additional user-defined field objects as new child nodes to corresponding parent nodes of the one or more additional user-defined field objects; generating the electronic form, using the form engine configured by the processor of the healthcare event management system, wherein the electronic form includes an ordered collection of form fields instantiated using the field objects, the ordered collection based at least on the relationships stored in the metabase including at least the meta-tables, the meta-relations, and the new meta-relations, the ordered collection of form fields being a set of visual elements;
controlling the display to automatically return and display, from the system interface to the user interface component via the network, the set of visual elements, wherein each visual element corresponds to a potential field value associated with the corresponding field object used to instantiate a corresponding form field responsive to the estimated cognitive level of the individual selected from the plurality of cognitive levels;
controlling the electronic form using the set of visual elements based at least on the relationships stored in the metabase to adopt or accommodate the expanded taxonomy; and
receiving, at the system interface, a selected visual element from the user interface component via the network, the selected visual element being from the set of visual elements.

12. The computer implemented method of claim 11, wherein the expanded taxonomy includes modifications to the set of visual elements of the electronic form.

13. The computer implemented method of claim 12, wherein the modifications to the set of visual elements of the electronic form includes at least one of a displayed font size, an object visual factoring size, and a selected visual theme.

14. The computer implemented method of claim 13, wherein the selected visual theme is modified through a substitution of a cascading style sheet (CSS).

15. The computer implemented method of claim 11, wherein the data set representative of the one or more electronic health records associated with the individual is retrieved using a domain specific query language.

16. The computer implemented method of claim 11, further comprising:
determining a group of target individuals to survey identified based on one or more common characteristics extracted from one or more electronic health records corresponding to each individual of the group of target individuals;
for each individual of the group of target individuals, generating one or more individualized electronic form using the metabase; and
controlling one or more user interface components, each corresponding to an individual of the group of target individuals, to render the one or more individualized electronic forms.

17. The computer implemented method of claim 11, wherein each of the new meta-relations includes a corresponding normalization factor, and the method further comprises:
receiving one or more data sets representative of responses from the electronic form;
traversing the metabase to identify one or more normalization factors associated with the set of visual elements utilized in generating the form;
applying the one or more normalization factors to transform the one or more data sets into a normalized data set; and
storing the normalized data set in a data storage maintaining response data sets normalized to a pre-expansion taxonomy of field objects.

18. The computer implemented method of claim 11, wherein the electronic form comprises a set of sequential interface screens; the method further comprising:
tracking one or more response characteristics of the individual as the interface renders each interface screens;
responsive to the tracked one or more response characteristics, re-determining the estimated cognitive level of the individual; and
responsive to a change in the estimated cognitive level of the individual, re-generating one or more remaining interface screens of the set of sequential interface screens of the electronic form.

19. The computer implemented method of claim 11, wherein the field object variations representative of variations of field objects that correspond to a plurality of cognitive levels include differing question strings appropriate for corresponding cognitive levels of the plurality of cognitive levels.

20. A computer-implemented method for dynamically generating an electronic form for receiving electronic data relating to a health care event at a user interface component, the electronic form adapted to provide a flexible meta-schema allowing an expanded taxonomy to be adopted or accommodated without modifications to applications that process the electronic form, the method comprising:
receiving a data set representative of one or more electronic health records associated with an individual;
determining an estimated cognitive level of the individual by parsing the data set;
providing a healthcare event management system including a storage device and a processor, the storage device having a database associated therewith to maintain the meta-schema and the processor configured to provide a form engine and a system interface;
establishing a communication link between the system interface and the user interface component over a network;
receiving, at the system interface from the user interface component via the network, a request to generate the electronic form using the form engine;
providing a dictionary of field objects in the database associated with the storage device of the healthcare event management system, wherein an instance of a field object is a form field, and wherein the form field is configured to receive a field value, the database structuring the dictionary of field objects into corresponding meta-tables and establishing meta-relations defining parent-child relationships between nodes representing the one or more field objects of the database, the meta-relations being maintained as corresponding foreign keys used to reference the corresponding field objects and the corresponding meta-tables, the database configured to have a dynamic number of columns that is dynamic according to a number of field objects in the dictionary;
adding one or more additional user-defined field objects in the database to accommodate an expanded taxonomy capturing field object variations representative of variations of field objects that correspond to a plurality of cognitive levels;
automatically maintaining, by the database, electronic representations of relationships between the field objects and the form fields, the one or more additional user-defined field objects representing the expanded taxonomy, the automatic maintaining including:
establishing, by the database, one or more new columns corresponding to the one or more additional user-defined field objects; and
establishing new meta-relations defining the one or more additional user-defined field objects as new child nodes to corresponding parent nodes of the one or more additional user-defined field objects; generating the electronic form, using the form engine configured by the processor of the healthcare event management system, wherein the electronic form includes an ordered collection of form fields instantiated using the field objects, the ordered collection based at least on the relationships stored in the database including at least the meta-tables, the meta-relations, and the new meta-relations, the ordered collection of form fields being a set of visual elements;
controlling the display to automatically return and display, from the system interface to the user interface component via the network, the set of visual elements, wherein each visual element corresponds to a potential field value associated with the corresponding field object used to instantiate a corresponding form field responsive to the estimated cognitive level of the individual selected from the plurality of cognitive levels;
controlling the electronic form using the set of visual elements based at least on the relationships stored in the database to adopt or accommodate the expanded taxonomy; and
receiving, at the system interface, a selected visual element from the user interface component via the network, the selected visual element being from the set of visual elements.
